(19) United States
(12) Patent Application Publication
(10) Pub. No.: US 2026/0198871 A1
(43) Pub. Date: Jul. 16, 2026

(54) IMAGING SYSTEMS, METHODS, AND APPARATUS THEREOF

(71) Applicant: UNITED IMAGING HEALTHCARE NORTH AMERICA, INC., Houston, TX (US)

(72) Inventors: Hongdi LI, Houston, TX (US); Yusheng LI, Houston, TX (US); Matthew DILSIZIAN, Houston, TX (US); Dongxu YANG, Houston, TX (US)

(73) Assignee: UNITED IMAGING HEALTHCARE NORTH AMERICA, INC., Houston, TX (US)

(21) Appl. No.: 19/182,543

(22) Filed: Apr. 17, 2025

Related U.S. Application Data
(60) Provisional application No. 63/745,305, filed on Jan. 14, 2025.

(51) Int. Cl. A61B 6/03; A61B 6/42

(52) U.S. Cl. CPC A61B 6/037; A61B 6/4241

(57) ABSTRACT

The present disclosure provides an imaging system comprising an imaging apparatus. The imaging apparatus may include a detector including detecting modules arranged along a circumference direction of the imaging apparatus and configured to form an accommodation space and a collimator including collimating modules arranged within the accommodation space along the circumference direction, the collimating modules including collimating units. The imaging apparatus may be configured to scan a target subject. During the scan of the target subject, at least a portion of the collimating units may be in an effective state for allowing radiation rays from the target subject to pass through and irradiate the detecting modules. The at least a portion of the collimating units may include multiple types of collimating units.

### CROSS-REFERENCE TO RELATED APPLICATIONS

This application claims priority to U.S. Provisional Patent Application No. 63/745,305, filed on Jan. 14, 2025, the contents of which are incorporated herein by reference.

### TECHNICAL FIELD

The present disclosure relates to medical imaging, and more particularly, relates to systems and methods for Single Photon Emission Computed Tomography (SPECT) using hybrid collimators.

### BACKGROUND

SPECT is a nuclear medicine imaging technique used to obtain images of organs or tissues of the human body through gamma rays produced by radioisotopes. In a SPECT system, a collimator is often used to collimate or shape a radiation field and define an incident angle of radiation rays reaching a detector, thereby ensuring that the acquired data by the detector is used to accurately reconstruct a distribution of a radiotracer inside a subject. Therefore, the collimator affects an image performance of the SPECT system, such as, a spatial resolution, a sensitivity, a field of view (FOV), etc.

Therefore, it is desired to provide systems and methods for SPECT using specially designed collimators (e.g., hybrid collimators), which can optimize trade-offs among the spatial resolution, the sensitivity, the FOV, etc., of the SPECT system, thereby improving the image performance of the SPECT system.

### SUMMARY

According to one or more embodiments of the present disclosure, an imaging system is provided. The imaging system may include an imaging apparatus. The imaging apparatus may include a detector including detecting modules arranged along a circumference direction of the imaging apparatus and configured to form an accommodation space, and a collimator including collimating modules arranged within the accommodation space along the circumference direction. The collimating modules may include collimating units. The imaging apparatus may be configured to scan a target subject. During the scan of the target subject, at least a portion of the collimating units may be in an effective state for allowing radiation rays from the target subject to pass through and irradiate the detecting modules, and the at least a portion of the collimating units include multiple types of collimating units.

In some embodiments, the multiple types of collimating units may be arranged on the same collimating module.

In some embodiments, the multiple types of collimating units may be arranged on different collimating modules.

In some embodiments, the multiple types of collimating units may have different aperture sizes.

In some embodiments, the multiple types of collimating units may include a first collimating unit and a second collimating unit, the first collimating unit may have a first aperture size, the second collimating unit may have a second aperture size smaller than the first aperture size. A first distance between the first collimating unit and a central axis of the imaging apparatus may be the same as a second distance between the second collimating unit and the central axis of the imaging apparatus, and the scan may be a single-FOV scan.

In some embodiments, the multiple types of collimating units may include a first collimating unit and a second collimating unit, the first collimating unit may have a first aperture size, the second collimating unit may have a second aperture size smaller than the first aperture size. A first distance between the first collimating unit and a central axis of the imaging apparatus may be different from a second distance between the second collimating unit and the central axis of the imaging apparatus, and the scan may be a dual-FOV scan.

In some embodiments, the dual-FOV scan may have a first FOV formed by the first collimating unit and a second FOV formed by the second collimating unit, the target subject may include a region of interest (ROI), the first FOV may cover the target subject, and the second FOV may coves the ROI.

In some embodiments, the dual-FOV scan may have a first FOV formed by the first collimating unit and a second FOV formed by the second collimating unit. The imaging system may further comprise a processing device configured to: obtain scan data collected by the detector during the scan; generate a target reconstruction image of the target subject by reconstructing the scan data, wherein the target reconstruction image includes a second portion corresponding to the second FOV and a first portion other than the second portion, the voxel size of voxels in the second portion is smaller than the voxel size of voxels in the first portion.

In some embodiments, the multiple types of collimating units may include a first collimating unit and a second collimating unit. The first collimating unit may have a first aperture size, and the second collimating unit may have a second aperture size smaller than the first aperture size. The imaging system may further comprise a processing device configured to: obtain scan data collected by the detector during the scan, the scan data includes first scan data corresponding to the first collimating unit and second scan data corresponding to the second collimating unit; generate an initial reconstruction image of the target subject based on the first scan data; and generate a target reconstruction image of the target subject by updating the initial reconstruction image based on the second scan data.

In some embodiments, the imaging system may further comprise a processing device configured to: obtain scan data collected by the detector during the scan, the scan data includes multiple data subsets corresponding to the multiple types of collimating units; generate a target reconstruction image of the target subject by reconstructing the scan data through an iterative process including iterations. A current iteration may include: for each type of collimating unit, generating an intermediate reconstruction image based on the data subset corresponding to the type of collimating unit and a combined reconstruction image of a previous iteration; generating a combined reconstruction image of the current iteration by combining the intermediate reconstruction images of the multiple types of collimating units based on weights of the multiple types of collimating units; proceeding to a next iteration or designating the combined reconstruction image of the current iteration as the target reconstruction image.

In some embodiments, the weight of a type of collimating unit may be determined based on a sensitivity map of the type of collimating unit.

In some embodiments, the weight of a type of collimating unit may be determined based on a scaling factor corresponding to the type of collimating unit, the scaling factor may be associated with the count of radiation events detected by the detector in the scan.

In some embodiments, the weights of the multiple types of collimating units may be determined based on scaling factors of the multiple types of collimating units, and the scaling factors may be determined by: obtaining candidate reconstruction images corresponding to candidate sets each of which includes candidate scaling factors of the multiple types of collimating units; determining a contrast recovery coefficient (CRC) and a signal-to-noise ratio (SNR) of each of the candidate reconstruction images; determining the scaling factors based on the CRC and the SNR of each of the candidate reconstruction images.

In some embodiments, during the scan of the target subject, the collimator may be caused to rotate relative to the detector around a central axis of the imaging apparatus by a preset step size one or more times.

In some embodiments, the preset step size may be determined such that each time the collimator rotates, projections of the at least a portion of the collimating units on the detecting modules may be shifted by a distance smaller than a bin size of the detector.

In some embodiments, the imaging system may further comprise a processing device configured to: determine a desired image resolution and a desired sensitivity of the imaging apparatus based on information relating to the target subject; determine reference values of structure parameters of the collimating units based on the desired image resolution and the desired sensitivity; and determine, based on the reference values of the structure parameters of the collimating units, target values of the structure parameters and counts of the different types of collimating units.

According to one or more embodiments of the present disclosure, a method for medical imaging is provided. The method for medical imaging may be implemented on a computing device having at least one processor and at least one storage device. The method may include obtaining scan data collected by a detector of an imaging apparatus during a scan of a target subject, and generating a target reconstruction image of the target subject by reconstructing the scan data. The scan data may be collected by the detector by detecting radiation rays from the target subject that pass through at least a portion of collimating units of a collimator of the imaging apparatus, the at least a portion of the collimating units may include multiple types of collimating units in different configurations.

In some embodiments, the scan data may include multiple data subsets corresponding to the multiple types of collimating units, and the generating a target reconstruction image of the target subject may include: for each type of collimating unit, generating an intermediate reconstruction image based on the data subset corresponding to the type of collimating unit and a combined reconstruction image of a previous iteration; generating a combined reconstruction image of the current iteration by combining the intermediate reconstruction images of the multiple types of collimating units based on weights of the multiple types of collimating units; proceeding to a next iteration or designating the combined reconstruction image of the current iteration as the target reconstruction image.

According to one or more embodiments of the present disclosure, an imaging system is provided. The imaging system may include an imaging apparatus. The imaging apparatus may include a detector including detecting modules arranged along a circumference direction of the imaging apparatus and configured to form an accommodation space, and a collimator including collimating modules arranged within the accommodation space along the circumference direction. The collimating modules may include collimating units. The imaging apparatus may be configured to scan a target subject. During the scan of the target subject, at least a portion of the collimating units may be in an effective state for allowing radiation rays from the target subject to pass through and irradiate the detecting modules, the collimator may be caused to rotate relative to the detector around a central axis of the imaging apparatus by a preset step size one or more times, and the preset step size may be determined such that each time the collimator rotates, projections of the at least a portion of the collimating units on the detecting modules are shifted by a distance smaller than a bin size of the detector.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, where like reference numerals represent similar structures throughout the several views of the drawings, and wherein:

FIG. 1 is a schematic diagram illustrating an imaging system according to some embodiments of the present disclosure;

FIG. 2 is a schematic diagram illustrating an exemplary imaging apparatus according to some embodiments of the present disclosure;

FIG. 3A is a schematic diagram illustrating a side view of an exemplary imaging apparatus from an axial direction according to some embodiments of the present disclosure;

FIG. 3B is a schematic diagram illustrating an unfolding view of the imaging apparatus shown in FIG. 3A along a circumference direction according to some embodiments of the present disclosure;

FIG. 4 is a schematic diagram illustrating an exemplary reception of radiation rays by detecting modules according to some embodiments of the present disclosure;

FIG. 5 is a schematic diagram illustrating an exemplary reception of radiation rays according to some embodiments of the present disclosure;

FIGS. 6A-6F are schematic diagrams illustrating side views of exemplary imaging apparatuses from an axial direction according to some embodiments of the present disclosure;

FIG. 7 is a schematic diagram illustrating an exemplary imaging apparatus according to some embodiments of the present disclosure;

FIG. 8 is a schematic diagram illustrating an exemplary imaging apparatus according to some embodiments of the present disclosure;

FIG. 9 is a block diagram illustrating an exemplary processing device according to some embodiments of the present disclosure;

FIG. 10 is a flowchart illustrating an exemplary process for generating a target reconstruction image according to some embodiments of the present disclosure;

FIG. 11A is a schematic diagram illustrating an exemplary process for generating a target reconstruction image according to some embodiments of the present disclosure;

FIG. 11B is a schematic diagram illustrating another exemplary process for generating a target reconstruction image according to some embodiments of the present disclosure;

FIG. 12 is a flowchart illustrating an exemplary process for determining scaling factors according to some embodiments of the present disclosure;

FIG. 13 is a flowchart illustrating an exemplary process for determining target values of structure parameters of a collimator according to some embodiments of the present disclosure;

FIGS. 14A-14C are schematic diagrams illustrating images indicating data in sinogram format according to some embodiments of the present disclosure;

FIGS. 15A-15F are schematic diagrams illustrating reconstruction images according to some embodiments of the present disclosure;

FIGS. 16A-16C are schematic diagrams illustrating reconstruction images according to some embodiments of the present disclosure;

FIGS. 17A-17D are schematic diagrams illustrating reconstruction images according to some embodiments of the present disclosure;

FIGS. 18A and 18B are schematic diagrams illustrating an exemplary process for super-sampling according to some embodiments of the present disclosure;

FIG. 19A is a schematic diagram illustrating an exemplary sampling pattern in sinogram space for a traditional sampling mode according to some embodiments of the present disclosure;

FIG. 19B is a schematic diagram illustrating an exemplary sampling pattern in sinogram space for a super-sampling mode according to some embodiments of the present disclosure;

FIG. 20 is a schematic diagram illustrating images indicating data in sinogram format according to some embodiments of the present disclosure;

FIGS. 21A-21D are schematic diagrams illustrating reconstruction images according to some embodiments of the present disclosure; and

FIGS. 22A-22F are schematic diagrams illustrating reconstruction images according to some embodiments of the present disclosure.

### DETAILED DESCRIPTION

In order to illustrate the technical solutions related to the embodiments of the present disclosure, a brief introduction of the drawings referred to in the description of the embodiments is provided below. Obviously, the drawings described below are only some examples or embodiments of the present disclosure. Those skilled in the art, without further creative efforts, may apply the present disclosure to other similar scenarios according to these drawings. Unless apparent from the locale or otherwise stated, like reference numerals represent similar structures or operations throughout the several views of the drawings.

It should be understood that the terms “system,” “device,” “unit,” and/or “module” used herein are one method to distinguish different components, elements, parts, sections, or assemblies of different levels in ascending order. However, the terms may be displaced by another expression if they achieve the same purpose.

As used in the disclosure and the appended claims, the singular forms “a,” “an,” and/or “the” may include plural forms unless the content clearly indicates otherwise. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may further include other steps or elements.

Unless otherwise defined, all technical and scientific terms used herein have the same meaning as commonly understood by those skilled in the art belonging to the present disclosure. The terms used herein in the specification of the present disclosure are for the purpose of describing specific embodiments only and are not intended to limit the invention. The term “and/or” as used herein includes any and all combinations of one or more of the relevant listed items.

An image performance (e.g., a resolution, a sensitivity, an FOV, etc.) of an imaging apparatus (e.g., a SPECT apparatus) is often affected by a detector and a collimator of the imaging apparatus. Conventional Nal (TI) detectors in the SPECT apparatus can achieve a resolution of approximately 2.9 to 3.5 millimeters (mm). Though Cadmium Zinc Telluride (CZT) detectors can achieve a resolution better than 0.5 mm, the CZT detectors are too costly for clinical whole-body imaging applications (e.g., the SPECT apparatus). To optimize trade-offs among the resolution, the sensitivity, the FOV, etc., of the imaging apparatus, multiple-pinhole collimators have proven effective in small animal SPECT imaging.

However, adapting the pinhole geometry for clinical systems poses challenges, as the clinical systems typically rely on minified projections to accommodate more pinholes, thereby increasing sensitivity. This minification, though beneficial for sensitivity, can reduce the resolution in reconstructed images.

To achieve optimal trade-offs between the resolution and sensitivity, an imaging system with a hybrid collimator is provided. The imaging system may include a detector including detecting modules arranged along a circumference direction of the imaging apparatus and configured to form an accommodation space. The hybrid collimator may include collimating modules arranged within the accommodation space along the circumference direction, and the collimating modules may include collimating units. The imaging system may be configured to scan a target subject. During the scan of the target subject, at least a portion of the collimating units may be in an effective state for allowing radiation rays from the target subject to pass through and irradiate the detecting modules, and the at least a portion of the collimating units may include multiple types of collimating units. For example, different types of collimating units may be in different configurations (e.g., large pinholes, small pinholes, etc.).

Since the hybrid collimator includes the multiple types of collimating units in different configurations, the imaging system can simultaneously collect scan data through multiple types of collimating units and combine advantages of the multiple types of collimating units. For example, the imaging system can simultaneously collect scan data through both the small pinholes with high resolution and the large pinholes with high sensitivity. This can achieve an optimal balance between the resolution and sensitivity, thereby improving the image performance of the imaging system.

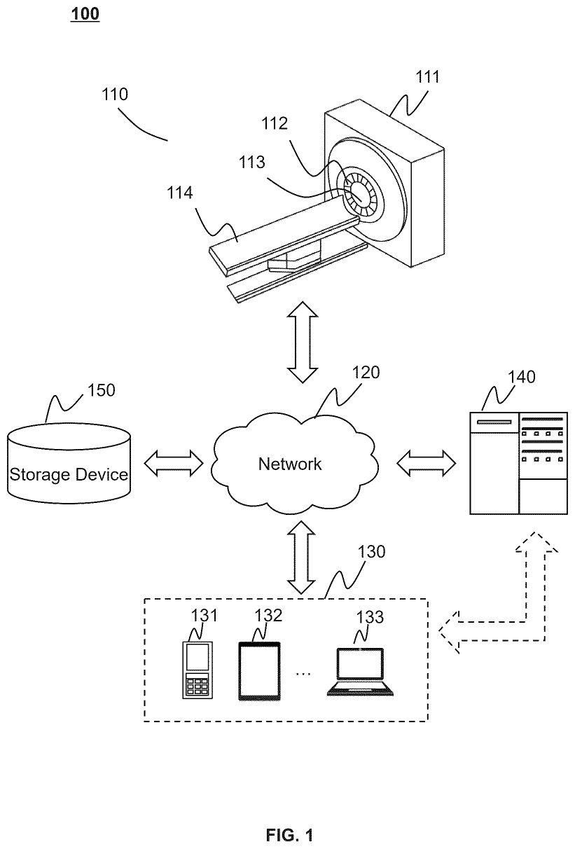

FIG. 1 is a schematic diagram illustrating an exemplary imaging system 100 according to some embodiments of the present disclosure.

As shown in FIG. 1, the imaging system 100 may include an imaging apparatus 110, a network 120, a terminal device 130, a processing device 140, and a storage device 150.

The imaging apparatus 110 may be configured to scan a target subject to obtain image data (e.g., projection data, images, etc.). In some embodiments, the imaging apparatus 110 may include a medical imaging apparatus, e.g., a single-photon emission computed tomography (SPECT) imaging apparatus, or other imaging apparatus, such as a computed tomography (CT) imaging apparatus, a positron emission tomography (PET), a SPET-CT imaging apparatus, a SPECT-MR imaging apparatus, etc.

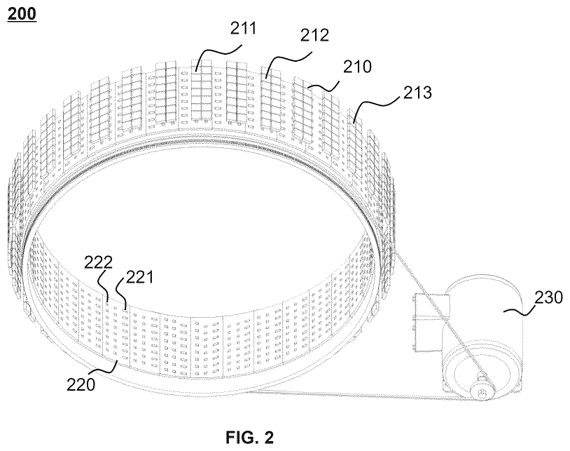

In some embodiments, the imaging apparatus 110 may include a gantry 111, a detector 112, and a scanning bed 114. A scanning region 113 may be provided for accommodating a subject to be scanned (also referred to as a target subject). The target subject may be placed on the scanning bed 114 and moved into the scanning region 113 to be scanned. The gantry 111 may provide support for other components (e.g., the detector 112) of the imaging apparatus 110. In some embodiments, the detector 112 may include one or more detecting modules arranged along a circumference direction of the imaging apparatus 110 and configured to form an accommodation space. The accommodation space may form the scanning region 113. A detecting module may include one or more detecting units arranged along a circumference direction perpendicular to an axial direction and/or the axial direction of the gantry 111. As used herein, the axial direction refers to a direction parallel to the long axis of the scanning bed 114. In some embodiments, each of the multiple detecting units may be configured to generate an electrical signal in response to detecting radiation rays. In some embodiments, each of the multiple detecting units or the detecting modules may be removable. It should be noted that the count of detecting modules in FIG. 1 is merely for illustration and does not limit the scope of the present disclosure, and a count of detecting modules may be multiple. A detecting unit may include a scintillator (such as a cesium iodide detector), a semiconductor, or the like. In some embodiments, the imaging apparatus 110 may further include a collimator (not shown in the figure). The collimator may include multiple collimating modules arranged within the accommodation space along the circumference direction of the imaging apparatus 110. The collimating modules may include collimating units, and at least a portion of the collimating units may include multiple types of collimating units. More descriptions regarding the collimator and/or detector may be found elsewhere in the present disclosure. See, e.g., FIGS. 2-8, and relevant descriptions thereof.

The network 120 may include any suitable network capable of facilitating an exchange of information and/or data for imaging apparatus 110. In some embodiments, at least one component of the imaging system 100 (e.g., the imaging apparatus 110, the terminal device 130, the processing device 140, the storage device 150) may exchange the information and/or data with the at least one other component of the imaging system 100 through the network 120. For example, the processing device 140 may obtain scan data from the imaging apparatus 110 through the network 120. In some embodiments, network 120 may include at least one network access point. For example, the network 120 may include a wired and/or wireless network access point (such as a base station and/or an Internet exchange point), and the at least one component of the imaging system 100 may be connected to the network 120 through the access point to exchange the data and/or information.

The terminal device 130 may communicate and/or connect with the imaging apparatus 110, the processing device 140, and/or the storage device 150. In some embodiments, the terminal device 130 may include a mobile device 131, a tablet computer 132, a laptop computer 133, or the like, or any combination thereof. For example, the mobile device 131 may include a mobile control handle, a personal digital assistant (PDA), a smartphone, or the like, or any combination thereof. In some embodiments, the terminal device 130 may include a display apparatus, such as a monitor. The display apparatus may be configured to display images or other information obtained by imaging, such as a medical image of a patient, a three-dimensional model, or an operation panel related to medical imaging. In some embodiments, the terminal device 130 may be a portion of the processing device 140.

The processing device 140 may be configured to the data and/or information obtained by the imaging apparatus 110, the terminal device 130, the storage device 150, or other components of the imaging system 100. For example, the processing device may be configured to perform one or more operations of the imaging method (e.g., a method for generating a target reconstruction image) disclosed in some embodiments of the present disclosure. In some embodiments, the processing device 140 may include a single server or a server group. The server group may include a centralized server group or a distributed server group. In some embodiments, the processing device 140 may include a local device or a remote device. For example, the processing device 140 may access the information and/or data from the imaging apparatus 110, the storage device 150, and/or the terminal device 130 through the network 120. As another example, the processing device 140 may be directly connected to the imaging apparatus 110, the terminal device 130, and/or the storage device 150 to access the information and/or data. As another example, the processing device 140 may be installed on the imaging apparatus 110. In some embodiments, the processing device 140 may be implemented on a cloud platform. For example, the cloud platform may include a private cloud, a public cloud, a hybrid cloud, a community cloud, a distributed cloud, an inter-cloud cloud, a multi-cloud, or the like, or any combination thereof.

The storage device 150 may be configured to store the data, instructions, and/or any other information. For example, the storage device 150 may store the data obtained by the imaging apparatus 110, the terminal device 130, and/or the processing device 140. In some embodiments, the storage device 150 may store the data and/or instructions used by the processing device 140 to execute or use to accomplish an exemplary method described in the present disclosure. In some embodiments, the storage device 150 may include a mass memory, a removable memory, a volatile read/write memory, a read-only memory (ROM), or the like, or any combination thereof. In some embodiments, the storage device 150 may be implemented on the cloud platform.

In some embodiments, the storage device 150 may be connected to the network 120 to communicate with the at least one other component of the imaging system 100 (e.g., the processing device 40, the terminal device 130). The at least one component of the imaging system 100 may access the data stored in the storage device 150 through the network 120. In some embodiments, the storage device 150 may be a portion of the processing device 140. In some embodiments, the processing device 140 and the storage device 150 may be integrated into the imaging apparatus 110.

It should be noted that the foregoing descriptions are merely provided for the purpose of illustration and are not intended to limit the scope of the present disclosure. For those skilled in the art, amendments and variations may be made under the teaching of the descriptions of the present disclosure. The features, structures, methods, and other characteristics of the exemplary embodiments described in the present disclosure may be combined in various manners to obtain additional and/or alternative exemplary embodiments. For example, the storage device 150 may be a data storage device that may include a cloud computing platform, such as public, private, community, and hybrid clouds. However, these amendments and variations do not depart from the scope of the present disclosure.

FIG. 2 is a schematic diagram illustrating an exemplary imaging apparatus 200 according to some embodiments of the present disclosure. As shown in FIG. 2, the imaging apparatus 200 may include a detector 210, a collimator 220, and a rotation transmission device 230.

The detector 210 may be configured to detect radiation rays (e.g., gamma rays) emitted from a subject to be scanned (also referred to as a target subject). For example, after a radioactive tracer is injected into the target subject, the radioactive tracer may decay to generate gamma rays. The gamma rays may be detected by the detector 210 and converted into electrical signals by the detector. The electrical signals may be further converted into digit signals which are used to generate an image of the target subject.

In some embodiments, the detector 210 may include multiple detecting modules (e.g., a detecting module 211, a detecting module 212, and a detecting module 213, etc.) arranged along a circumference direction of the imaging apparatus 200 to form an accommodation space. For example, the multiple detecting modules may be arranged along the circumference direction to form a cylindrical structure.

In some embodiments, each of the multiple detecting modules may include multiple detecting units arranged along the circumference direction of the imaging apparatus and/or an axial direction perpendicular to the circumference direction. A detecting unit may be a basic unit of the detector for detecting radiation rays, and the detecting unit may refer to the smallest unit of the detector that may independently detect particles or radiation rays, and generate an electrical signal. The multiple detecting units may be packaged to form a detecting box, i.e., a detecting module. The detecting units in each detecting module may be arranged along the circumferential direction and/or the axial direction perpendicular to the circumferential direction.

In some embodiments, each detecting unit may be made of any material that can absorb radiation rays and emit a portion of the absorbed radiation rays as light. For example, a detecting unit may be made of, for example, bismuth germanium oxide (BGO), lutetium oxyorthosilicate (LSO), lutetium-yttrium oxyorthosilicate (LYSO), lutetium-gadolinium oxyorthosilicate (LGSO), gadolinium oxyorthosilicate (GSO), yttrium oxyorthosilicate (YSO), barium fluoride, sodium iodide, cesium iodide, lead tungstate, yttrium aluminate, lanthanum chloride, lutetium-aluminum perovskite, lutetium disilicate, lutetium aluminate, lutetium iodide, thallium bromide, or the like, or any combination thereof. Different detecting units may be made of the same material or different materials.

In some embodiments, each of the multiple detecting modules may be detachable for efficiently adding, removing, and/or replacing the detecting modules from the imaging apparatus 200. In some embodiments, each of the multiple detecting units may be detachable for efficiently adding, removing, and/or replacing the detecting units from the imaging apparatus 200.

Compared with an arrangement of a single detecting module along a certain position of the circumference direction, an arrangement of the multiple (two or more) detecting modules along the circumference direction may increase effective detecting areas of the detector. For example, the multiple detecting modules may work simultaneously to obtain data from different angles, thereby improving a resolution, enhancing a sensitivity, and improving a redundancy and reliability. It should be noted that the count of detecting modules in FIG. 2 is merely for illustration and does not limit the scope of the present disclosure, and a count of detecting modules may be multiple.

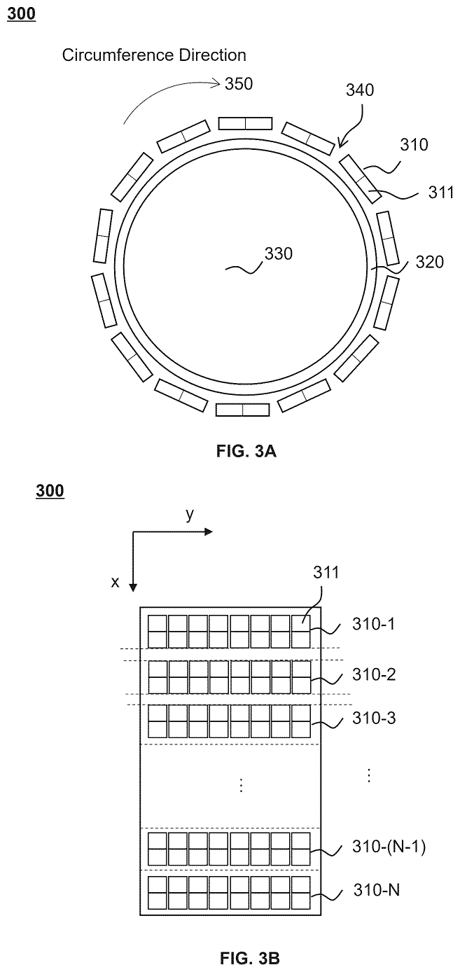

Merely by way of example, FIG. 3A is a schematic diagram illustrating a side view of an exemplary imaging apparatus 300 from an axial direction according to some embodiments of the present disclosure. As shown in FIG. 3A, the imaging apparatus 300 may include a detector and a collimator 320, and the detector may include multiple detecting modules 310. The multiple detecting modules 310 may be arranged along a circumference direction denoted by an arrow 350 in FIG. 3A. It should be noted that the count of detecting modules in FIG. 3A is merely for illustration and does not limit the scope of the present disclosure, and a count of detecting modules may be multiple.

The circumference direction refers to a direction along an edge of a circle, for example, as shown by the arrow 350 in FIG. 3A. The circumference direction of the imaging apparatus may be perpendicular to the axial direction of the imaging apparatus.

In some embodiments, the multiple detecting modules may be arranged at intervals. A space between adjacent detecting modules arranged at intervals may be referred to as a gap. That is, the gap may be involved between two adjacent detecting modules among the multiple detecting modules. In some embodiments, a length of the gap between the two adjacent detecting modules among the multiple detecting modules may be in a range of 2 to 4 centimeters (cm). In some embodiments, the length of the gap between the two adjacent detecting modules among the multiple detecting modules may be in a range of 2 to 6 cm. In some embodiments, the length of the gap between the two adjacent detecting modules among the multiple detecting modules may be in a range of 2 to 8 cm. In some embodiments, the length of the gap between the two adjacent detecting modules among the multiple detecting modules may be in a range of 2 to 10 cm. In some embodiments, the length of the gap between the two adjacent detecting modules among the multiple detecting modules may be in a range of 2 to 15 cm. In some embodiments, the length of the gap between the two adjacent detecting modules among the multiple detecting modules may be in a range of 2 to 20 cm. As used herein, the length of the gap may refer to a distance between two close/neighbor edges of the two adjacent detecting modules along the circumference direction. The distance between two close edges of the two adjacent detecting modules along the circumference direction may be a straight-line distance, a minimum distance, a minimum arc length, etc., connecting the two close edges of the two adjacent detecting modules.

For example, as shown in FIG. 3A, a space pointed by an arrow 340 between two adjacent detecting modules in FIG. 3A may be the gap. As another example, FIG. 3B is a schematic diagram illustrating an unfolding view of the imaging apparatus 300 shown in FIG. 3A along a circumference direction according to some embodiments of the present disclosure. An X direction in FIG. 3B may correspond to the circumference direction in FIG. 3A, and a Y direction may correspond to the axial direction. As shown in FIG. 3A and FIG. 3B, multiple detecting modules 310-1, 310-2, 310-3 . . . 310-N may be arranged along the circumference direction. Each of the multiple detecting modules 310-1, 310-2, 310-3, . . . 310-(N-1), 310-N, may include multiple detecting units 311. A space between two adjacent detecting modules (e.g., the detecting modules 310-1 and 310-2) may be a gap between the two adjacent detecting modules (e.g., the detecting modules 310-1 and 310-2), such as the space defined by adjacent dotted lines as shown in FIG. 3B. It should be noted that the count of detecting modules in FIG. 3B is merely for illustration and does not limit the scope of the present disclosure, and a count of detecting modules may be multiple.

In some embodiments, the length of the gap between any two adjacent detecting modules along the circumference direction in the imaging apparatus 200 may be the same or different.

In some embodiments, the detector 210 may correspond to a bin size. The bin size refers to the size of the smallest unit of data acquisition or measurement in the detector 210. For example, the bin size is equal to the width of a detecting unit of the detector 210. In some embodiments, the bin size may relate to a resolution (or an imaging resolution) of the detector 210 or the imaging apparatus 200. Exemplary resolutions may include a spatial resolution, an energy resolution, a temporal resolution, or the like, or any combination thereof. For example, a smaller bin size may correspond to more detailed data and finer resolution (e.g., smaller pixels or narrower energy ranges), and a larger bin size results in less detailed data and lower resolution.

In some embodiments, the accommodation space may be a cylindrical-like three-dimensional space (also referred to as a cylinder), and the axial direction of the cylinder (i.e., the axial direction of the imaging apparatus) may be perpendicular to the circumference direction of the cylinder. For example, as shown in FIG. 3A, an accommodation space 330 may be surrounded by the multiple detecting modules 310 arranged along the circumference direction.

In some embodiments, the collimator 220 may include collimating modules arranged within the accommodation space along the circumference direction and may be configured to rotate around an axis of the accommodation space that is perpendicular to the circumference direction.

The collimator 220 is a component of the imaging apparatus 200 configured to constrain transmission path(s) of radiation rays. In some embodiments, the collimator 220 may be made of a material with a relatively large attenuation coefficient for the radiation rays (e.g., a dense metallic material such as lead, tungsten, uranium, etc.). In some embodiments, the collimator 220 and the detector 210 may be arranged concentrically. The collimator 220 may be arranged between the center of the accommodating space and the detector 210. Further, the collimator 220 may be closer to the detector 210 than the center of the accommodating space.

In some embodiments, the collimator 220 may include multiple collimating modules arranged along the circumference direction of the detector 210, or the accommodating space, or the imaging apparatus 200.

In some embodiments, at least one of the multiple collimating modules may be detachable from the collimator 220 or the imaging apparatus 200. For example, the multiple collimating modules may be connected with a support of the collimator 220 via a detachable connection, such as a clamping connection, a screw connection, a rivet connection, a hinge connection, or the like, or any combination thereof. Via the detachable connection, one or more collimating modules may be detachable from the collimator 220 or added to the collimator 220, thereby achieving the replacement of the one or more collimating modules.

In some embodiments, one (or each) of the collimating modules may include multiple collimating units. The multiple collimating units may be arranged along the circumference direction of the detector 210, or the accommodating space, or the imaging apparatus 200. A collimating unit may be a basic unit of the collimator. The multiple collimating units in a collimating module may be working independently. For example, when one of the multiple collimating units in the collimating module working or operating, others of the multiple collimating units in the collimating module may be not working. In some embodiments, the multiple collimating units in the collimating module may be packaged to form a collimating box, i.e., the collimating module. In some embodiments, the collimating box may include a shell for packing the multiple collimating units. The shell of the collimating box may be made of a material with a lower attenuation coefficient of the material of the collimating unit. For example, the shell of the collimating box may be made of a plastic, a rubber, a carbon fiber, or the like, or any combination thereof.

Each of the collimating units in a collimating module may include a main body and one or more holes (or apertures) arranged on the main body. The main body may include a material with a high attenuation coefficient. In some embodiments, different collimating units may include different main bodies. In some embodiments, the different main bodies corresponding to different collimating units may be an integrated body. The collimating module may include multiple holes arranged on the integrated body. A hole (or aperture) may be configured to pass through radiation ray(s) and define an incident direction of the radiation ray(s) passing through the hole. For example, the hole may be a through hole. In some embodiments, the hole may include a hole canal. A radiation ray whose incident direction is parallel to an extending direction of the hole canal may pass through the hole canal. A radiation ray whose incident direction is not parallel to the extending direction of the hole canal may be absorbed by the main body. Therefore, a collimating unit may define an incident direction of the radiation ray(s) passing through the hole(s) in the collimating unit. In some embodiments, each hole in the collimating module may be regarded as one collimating unit.

In some embodiments, according to the shape of the hole(s) in a collimating unit, the collimating unit may include a pinhole, a parallel hole, a convergent hole, a fan-beam hole, etc. In some embodiments, the shape of the hole may include a circle, an oval, a polygon, etc.

In some embodiments, the collimator 220 may include multiple types of collimating units. For example, different types of collimating units may have different configurations. As used herein, a configuration of a collimating unit may be defined by one or more structure parameters. Exemplary structure parameters of the collimating unit may include an aperture size (e.g., an aperture diameter) of a hole in the collimating unit, a hole channel length, an opening angle, a count of the hole(s) in the collimating unit, a distribution or arrangement of the hole(s) in the collimating unit, a distance between the collimating unit and the center of the accommodating space, or the like, or any combination thereof. The aperture size of the hole may include a diameter, an edge length, a diagonal, etc., of the hole. In some embodiments, the aperture size may be within a length range. For example, the aperture size may be within a range of 0.1 millimeters (mm) to 10 mm. As another example, the aperture size may be within a range of 0.5 mm to 5 mm. As still another example, the aperture size may be within a range of 1 mm to 3 mm. As yet another example, the aperture size may be within a range of 1 mm to 2 mm. The hole channel length refers to a length of a hole canal of the hole. For a pinhole, a fan-beam hole, or a convergent hole, the hole canal and/or the extension of the hole may be regarded as a cone, and the opening angle may also be referred to as a taper angle that is an angle between the two generatrixes of the axis section (passing through the axis of the cone) of the cone. The distribution or arrangement of the hole(s) in the collimating unit may refer to positions of the hole(s) in the collimating unit. For example, the hole(s) in the collimating unit may be arranged in the main body of the collimating unit regularly, for example, in a form of a matrix including M rows and N columns. As another example, the hole(s) in the collimating unit may be arranged in the main body of the collimating unit irregularly.

As used herein, different configurations of two collimating units may refer to that at least one of the one or more structure parameters of the two collimating units may be different. For example, the multiple types of collimating units may have different aperture sizes. Merely by way of example, as shown in FIG. 2, the collimator 220 may include a first collimating unit 221 and a second collimating unit 222. The first collimating unit 221 may include a hole that has a first aperture size. The second collimating unit 222 may include a hole that has a second aperture size. The second aperture size may be smaller than the first aperture size, such that the first collimating unit 221 and the second collimating unit 222 have different configurations. Correspondingly, the hole that has the first aperture size may be also referred to as a large hole, and the hole that has the second aperture size may be also referred to as a small hole. The same configuration of two collimating units may refer to that each of the one or more structure parameters of the two collimating units is the same.

In some embodiments, one of the collimating modules may include a same type of collimating units. For example, one collimating module may include multiple first collimating units. In some embodiments, one of the collimating modules may include different types of collimating units. For example, one collimating module may include at least one first collimating unit and at least one second collimating unit.

In some embodiments, the multiple collimating modules may be rotated around a central axis of the imaging apparatus 200 (e.g., a central axis of the accommodation space that is parallel to the axial direction of the imaging apparatus 200) perpendicular to the circumference direction to change the positions of the multiple collimating modules along the circumference direction. Each of the multiple collimating units may be switched between an effective state and an invalid state via a rotation of the collimator 220 relative to the detector 210 around the central axis of the accommodation space. The rotation of the collimator 220 relative to the detector 210 refers to that only the collimator 220 rotates or the collimator 220 and the detector 210 rotate at different manner. For example, when the collimator 220 rotates around the central axis of the accommodation space, the detector 210 may be still. As another example, when the collimator 220 rotates around the central axis of the accommodation space, the detector 210 may rotate at a first rotation speed different from a second rotation speed of the collimator 220.

In some embodiments, for each of the multiple collimating modules, there is a distance between the central axis of the imaging apparatus 200 and the collimating module (or collimating unit(s) on the collimating module). For example, the distance between the central axis of the imaging apparatus 200 and the collimating module (or the collimating unit(s) on the collimating module) may include a distance between the central axis of the imaging apparatus 200 and a central point on a first surface of the collimating module (or the collimating unit(s)) close to the central axis, a distance between the central axis of the imaging apparatus 200 and a central point on a second surface of the collimating module (or the collimating unit(s)) far away from the central axis, a distance between the central axis of the imaging apparatus 200 and a geometric center point of the collimating module, a distance between the central axis of the imaging apparatus 200 and a geometric center point of one of the collimating unit(s), or the like, or any combination thereof. For ease of description, the distance between the central axis of the imaging apparatus 200 and the collimating module (or the collimating unit(s) on the collimating module) may be the distance between the central axis of the imaging apparatus 200 and the central point on the first surface of the collimating module (or the collimating unit(s)) close to the central axis.

It should be noted that the distance between the central axis of the imaging apparatus 200 and the central point on the first surface of the collimating module (or the collimating unit(s)) close to the central axis is merely provided for illustration, and is not intended to limit the scope of the present disclosure.

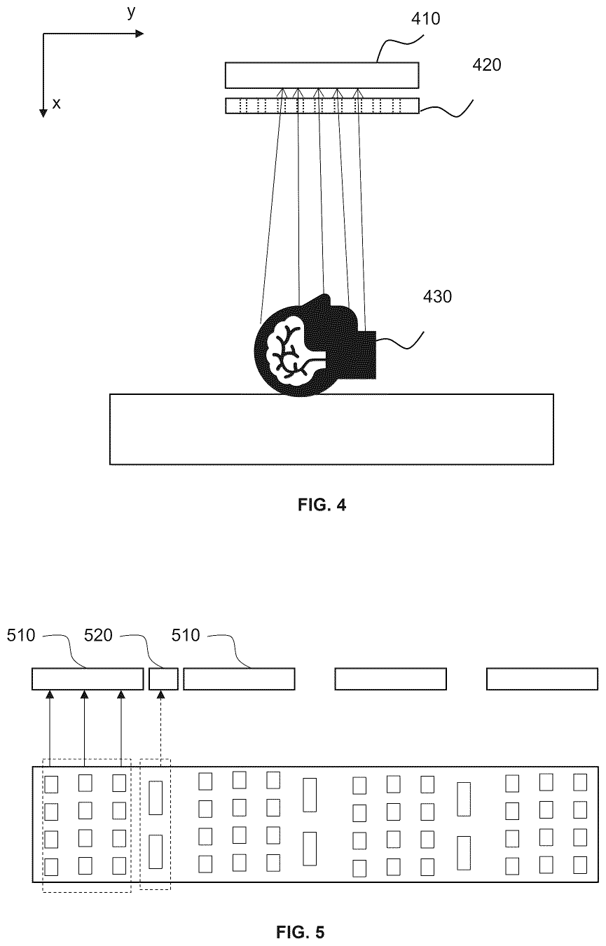

When a collimating unit is in the effective state, a projection of the collimating unit on the detector 210 may be located within a detecting module of the detector 210, and a radiation ray passing through the collimating unit may irradiate on the detecting module. That is, the radiation ray passing through the collimating unit may be able to be received by the detecting module. The effective state may also be referred to as an operating state or working state. As shown in FIG. 4, a target subject 430 may be located within the accommodation space. When the radiation rays emitted from the target subject 430 are able to pass through the holes in a collimating unit 420 to reach a detecting module 410, the collimating unit 420 may be in the effective state. It should be noted that the count of detecting modules in FIG. 4 is merely for illustration and does not limit the scope of the present disclosure, and a count of detecting modules may be multiple.

When a collimating unit is in the invalid state, a projection of the collimating unit on the detector 210 may be located within a gap that is between two adjacent detecting modules of the detector 210, and a radiation ray passing through the collimating unit may irradiate on the gap. That is, the radiation ray passing through the collimating unit may be not able to be received by any detecting module.

The rotation of the collimator 220 (e.g., the collimating module) relative to the detector 210 around the central axis of the accommodation space may change the positions of the multiple collimating units in the collimating module relative to the detector 210 along the circumference direction. When a collimating unit is rotated around the central axis of the accommodation space to a position corresponding to a gap between two adjacent detecting modules, the radiation ray passing through the collimating unit may not be able to be detected by a detecting module, thus the collimating unit may be in the invalid state. When the collimating unit is rotated around the central axis of the accommodation space to a position corresponding to the detecting module, a radiation ray passing through the collimating unit may reach and be detected by the detecting module, thus the collimating unit may be in the effective state. As described herein, the position of a collimating unit corresponding to the position of a detecting module may refer to a position where a projection of the collimating unit along the radial direction of the accommodating space on a plane (e.g., the circumferential surface where the detector is located) is located within a projection of the detecting module on the plane along the radial direction of the accommodating space. The position of a collimating unit corresponding to the position of a gap between two adjacent detecting modules may refer to a position where a projection of the collimating unit along the radial direction of the accommodating space on a plane (e.g., the circumferential surface where the detector is located) is located within a projection of the gap between two adjacent detecting modules on the plane along the radial direction of the accommodating space.

In some embodiments, the multiple collimating units in a collimating module may be composed of a first portion and a second portion. When the first portion of the multiple collimating units are in the effective state, the second portion of the multiple collimating units may be in the invalid state; when the first portion of the multiple collimating units is in the invalid state, the second portion of the multiple collimating units may be in the effective state.

In some embodiments, a collimating unit in the first portion and a collimating unit in the second portion may be changeable. For example, the collimating unit may be switched between the effective state and the invalid state by driving the collimating module to move along the circumference direction, and the collimating unit may be transformed in the first portion and the second portion according to the switching between the effective state and the invalid state. By driving the collimating module to rotate along the circumference direction, the position of the collimating unit may be changed, thereby causing the collimating unit to switch between the effective state and the invalid state. As a further example, when a collimating module is rotated by a certain angle, a collimating unit A in collimating module 1 may be in the effective state, a collimating unit B in collimating module 1 may be in the invalid state, and a collimating unit C may be in the effective state, then the collimating units A and C may be the collimating units in the first portion, and the collimating unit B may be a collimating unit in the second portion. When the collimating module is further rotated by a certain angle, the collimating unit A may be switched to the effective state, the collimating unit B may be switched to the effective state, and the collimating unit C may be switched to the invalid state, then the collimating units A and B may be the collimating units in the first portion, and the collimating unit C may be a collimating unit in the second portion.

In some embodiments, projections, along a radial direction of the accommodating space, of any two of the multiple collimating units on a plane where the detector is located may be independent. Independence of the projections may refer to that there is no overlapped space between the projections, along the radial direction of the accommodating space, of any two of the multiple collimating units on the plane where the detector is located.

In some embodiments, the projections, along the radial direction of the accommodating space, of any two of the multiple collimating units on the plane where the detector is located may overlap each other. A detection efficiency of the detector may be improved when the projections of any two of the multiple collimating units on the plane along the radial direction of the accommodating space are overlapped. The overlapped projected images may be separated and reconstructed through an image processing algorithm. The image processing algorithm may refer to a series of preset determination operations configured to process input data (the overlapped projected images herein) to generate a desired output (such as a separated image or a reconstructed image), and the specific image processing algorithm may include an image optimization technique or a machine learning model, etc.

In some embodiments, the independence of the projections of any two collimating units on the detector may make the radiation rays passing through the two collimating units not interfere with each other. Alternatively, the projections of any two collimating units along the radial direction on the plane where the detector is located may be non-overlapped. As used herein, the term “projection” of a component (e.g., the collimating unit, the collimating module, the detecting module, the gap between two adjacent detecting modules) may be a region obtained by projecting the shape of the component onto a plane with a beam of light along a reference direction. The shape of the gap between the two adjacent detecting modules may be defined by edges of the two adjacent collimating modules (e.g., a visible ray or a virtual line). The direction of the beam of light may be parallel to the reference direction. For example, the projection of the collimating unit on a plane where the detector is located along the radial direction of the accommodating space may include a region formed by projecting the shape of the collimating unit onto the plane with a beam of light with a direction parallel to the radial direction. In some embodiments, the plane where the detecting module is located may include a plane passing through a geometric center of the detecting module and perpendicular to the radial direction. In some embodiments, the detecting module may be curved-shape, then the plane where the detecting module is located refers to a plane where a cut surface of the detecting module at the geometric center is located. In some embodiments, the plane where the detecting module is located may include the plane defined by the surface of the detecting module.

By setting the projections of any two collimating units of the multiple collimating units on the detector along the radial direction to be independent of each other, measurement accuracy may be improved, signal interference may be reduced, and imaging quality may be improved.

In some embodiments, each of the collimating modules may correspond to one of the detecting modules. In other words, the count or number of the collimating modules may be less than or equal to the count or number of the detecting modules. A radiation ray passing through a collimating unit in the effective state in a collimating module may be irradiated on the detecting module corresponding to the collimating module; and a radiation ray passing through a collimating unit in the invalid state in the collimating module may be irradiated in a gap between the detecting module and the adjacent detecting module.

In some embodiments, the gap between two adjacent detecting modules may include a shielding module. The radiation ray passing through a collimating unit in the invalid state in the collimating module and reaching the gap between two adjacent detecting modules may be absorbed by the shielding module. As shown in FIG. 5, the radiation rays passing through the collimating unit in the invalid state may further be irradiated on a shielding module 520 between two adjacent detecting modules 510. In some embodiments, the shielding module may be a component made of a dense material (e.g., lead or tungsten, etc.) that has a high attenuation coefficient. In some embodiments, the material of the shielding module may be the same as the material of the main body of the collimating unit. In some embodiments, the material of the shielding module may be different from the material of the main body of the collimating unit. It should be noted that the count of detecting modules in FIG. 5 is merely for illustration and does not limit the scope of the present disclosure, and a count of detecting modules may be multiple.

In some embodiments, the effective state and/or the invalid state of a collimating unit may be changed by adding/removing a shielding module to block/unblock the collimating unit. When the shielding module is added to block the collimating unit, the collimating unit is in the invalid state. When the shielding module is removed to unblock the collimating unit, the collimating unit is in the effective state.

In some embodiments, when the first portion of the multiple collimating units in a collimating module is in an edge region of the collimating module and in the effective state, the second portion of the multiple collimating units in the collimating module may be in a middle region of the collimating module and in the invalid state, and the projection of the second portion of the multiple collimating units along a radial direction on the detector may be located within one single gap between the detecting module and the adjacent detecting module.

In some embodiments, when the first portion of the multiple collimating units is in a middle region of the collimating module and is in the effective state, the second portion of the multiple collimating units may be in two edge regions of the collimating module, the projection of the second portion of the multiple collimating units along the radial direction on the detector may be located within two gaps each of which is between the detecting module and the adjacent detecting module.

In some embodiments, a projection of a collimating unit in the effective state along a radial direction on the detector may be located within a detecting module.

As used herein, the projection located within the detecting module may refer to a region formed by the projection may be within the region where the detecting module is located.

In some embodiments, the length of the gap between two adjacent detecting modules along the circumference direction may exceed a length of a collimating module along the circumference direction.

In some embodiments, the length of the gap along the circumference direction may exceed a length of the second portion of the multiple collimating units in the invalid state along the circumference direction.

In some embodiments, when a collimating unit is in the effective state, the collimator 220 may be driven to rotate an angle based on a sampling rate of the imaging system 100 (e.g., the imaging apparatus 110, the imaging apparatus 200).

A sampling rate of an imaging system may be a spatial frequency where the detector collects data. The sampling rate may reflect the ability of the detector to discriminate different radiation rays in space.

In some embodiments, by driving the collimator 220 to rotate to precisely micro-move a collimation angle, the sampling rate may be increased, and the imaging quality may be improved. For example, the collimator may be driven to rotate a plurality of times by a small magnitude (e.g., half the width of a detecting unit or the bin size) to collect multi-angle information. A micro-movement of a collimating unit to adjust the collimation angle of the collimating unit may further eliminate a quantization error caused by a detector pixelation. For example, rotating the collimator 220 with half the width of a detecting unit may be equivalent to reducing a half the size of a detecting unit along a rotation direction. The position of the collimating unit in the effective state relative to the detecting module may affect the sampling rate of the detecting module. For example, by driving the collimator to rotate the plurality of times, the positions of the different detecting units in the detecting module may be adjusted relatively to the position of the detecting module. The projection of the collimating unit in the effective state along the radial direction towards the plane where the detecting module is located may be located within an edge region or a center region of the detecting module by rotating the collimator around the axial direction. When the projection of the collimating unit in the effective state along the radial direction towards the plane where the detecting module is located may be located within the center region of the detecting module, since the radiation lines passing through the collimating units are received by the detecting module over a relatively large region, the sampling rate of the detecting module may be increased. In some embodiments, the detecting module may include multiple scintillators, each of the multiple scintillators may correspond to a detecting unit, and a desired sampling rate may be satisfied by adjusting a relative position relationship between the holes in the collimating units in the effective state and the multiple scintillators. If the radiation rays passing through the holes in a collimating unit in the effective state are detected by each of the multiple scintillators in the detecting module, the sample rate may be high; and if the radiation rays passing through the holes in a collimating unit in the effective state are detected by a portion of the multiple scintillators in the detecting module, the sample rate may be low.

In some embodiments, by obtaining a relationship between the sampling rate and an adjustment angle, an adjustment angle at the desired sampling rate may be determined based on the relationship between the sampling rate and an adjustment angle, thereby achieving needs at different sampling rates.

In some embodiments, the multiple collimating modules may be rotated around the central axis of the accommodation space with the rotation of the collimator 220 synchronously. In other words, when the collimator 220 rotates around the central axis of the accommodation space, the multiple collimating modules may rotate synchronously around the central axis of the accommodation space. In some embodiments, the multiple collimating modules may rotate around the central axis of the accommodation space independently. For example, when one of the multiple collimating modules rotates around the central axis of the accommodation space and the other one of the multiple collimating modules may not rotate.

In some embodiments, during the scan of the target subject, the collimator 220 may be caused to rotate relative to the detector 210 around the central axis of the imaging apparatus 200 by a preset step size one or more times. The preset step size refers to a rotation amplitude or angle of the collimator 220 relative to the detector 210 around the central axis of the imaging apparatus 200.

In some embodiments, the multiple collimating modules may be rotated around the central axis of the accommodation space through the rotation transmission device 230.

The rotation transmission device 230 may be configured to drive the collimator 220 to rotate. In some embodiments, the rotation transmission device 230 may include a rotating support and a driving component. The multiple collimating modules may be arranged on the rotating support, and the driving component may be configured to drive the rotating support to rotate.

The rotating support may be configured to hold and support the multiple collimating modules in the collimator 220. In some embodiments, the rotating support may be rotatably connected with the gantry of the imaging apparatus 200.

In some embodiments, the rotating support may be made of metal or other robust material. The rotating support may be driven by the driving component to rotate. For example, the rotating support may include a turntable and a collimator supporting structure. The collimator supporting structure may be connected to the turntable, the turntable may be capable of rotating under the driving component, and the collimator supporting structure may be configured to support and fix the collimator to cause the collimator to rotate under the driving of the turntable.

The driving component may be a component configured to drive the rotating support to rotate. In some embodiments, the driving component may include a motor and a transmission element. In some embodiments, the motor may include a stepper motor, a servo motor, or the like, or a combination thereof. The transmission element may include a gear transmission, a belt transmission, or the like, or a combination thereof. The motor may drive the rotating support to rotate through the transmission element. For example, the motor may include a rotation shaft (e.g., a rotor) connected with the transmission element. The motor may drive the rotation shaft to rotate and the rotation of the rotation shaft may drive the rotation of the rotating support to rotate.

In some embodiments, the rotation transmission device 230 may further include a positioning component configured to determine positions of the collimating modules in the collimator 220. A precise position of the collimating module in space may be monitored and determined by the positioning component for obtaining high quality imaging results.

In some embodiments, the positioning component may be implemented through a magnetic scale, an optical encoder, a Hall effect sensor, a voltage-based sensor, or a vision system (e.g., by capturing an image and tracking a position of the collimating module through image processing and algorithmic recognition). For example, the turntable may be equipped with a high-precision positioning component (e.g., a magnetic scale), which may be configured to measure an angle of rotation of the turntable in real time to reach a purpose of precise control.

In some embodiments, the motor and the positioning component may cooperate to cause the collimator to rotate a plurality of times with a small amplitude (e.g., half the width of a detecting unit or the bin size) to collect information from multiple angles and improve the quality of image reconstruction. In some embodiments, the motor and the positioning component may be used for calibration of the detector.

In some embodiments, the processing device 140 may cause the collimator 220 to rotate around the axis of the imaging apparatus 200 to cause target collimating unit(s) of the multiple collimating units in each collimating module of the collimator 220 to be in an effective state, so as to obtain scan data of the target subject. Then one or more images of the target subject may be reconstructed based on the scan data of the target subject. In some embodiments, the processing device 140 may determine the target collimating unit(s) in each collimating module of the collimator 220 that needs to be in the effective state according to imaging requirements on one or more imaging parameters. For example, if the target subject includes the heart of the human body, the target collimating unit(s) may correspond to an FOV whose center is misalign with a center of a circumference plane where the target collimating unit(s) are located. As another example, if the target subject includes the body of a patient, the target collimating unit(s) may correspond to a maximum FOV. In some embodiments, the processing device 140 may rotate the collimator 220 with a rotation angle to cause the target collimating unit(s) in the effective state. The rotation angle may be determined based on position(s) of the target collimating unit(s) are located. The position of the target collimating unit(s) may be determined based on a positioning component.

In some embodiments, during a scan of the target subject, collimating units in the multiple collimating modules of the collimator 220 may be in the effective state simultaneously. The configuration of the collimating units in the multiple collimating modules that are in the effective state simultaneously may be related to an imaging requirement. The imaging requirement may be defined by one or more imaging parameters, such as, an FOV, a sensitivity, a resolution, a detecting rate, etc., of the imaging apparatus 200. Accordingly, the configuration of each collimating unit may be designed according to an imaging requirement. For example, the sensitivity may be determined based on the aperture size of the hole and a distance between the collimator 220 and a subject to be scanned (or the central axis of the imaging apparatus 200 (e.g., the central axis of the accommodation space)). As another example, the resolution may be determined based on the aperture size of the hole and the opening angle. As a further example, an FOV corresponding to a relatively large aperture size of holes may be larger than an FOV corresponding to a smaller aperture size of holes because the large aperture size allows more rays to pass through and may form a wide FOV. At the same time, the more radiation rays passing through the holes with the larger aperture size to reach the detector 210, the more sensitivity may be improved; while the holes with the smaller aperture size may precisely limit a range of path of the radiation rays that pass through the collimator 220, which may improve the resolution.

In order to maximize imaging performance and achieve an optimal balance between the resolution and the sensitivity, the present disclosure proposes a hybrid collimator. In some embodiments, a collimator including collimating units with different configurations may be referred to as a hybrid collimator. In some embodiments, at least a portion of the collimating units of a hybrid collimator may be in the effective state simultaneously during the scan, wherein the at least a portion of the collimating unit may have different configurations. In other words, collimating units in different configurations are in the effective state simultaneously for collecting scan data during the scan.

For illustration purposes, pinholes are described herein as exemplary collimating units, which are not intended to limit the scope of the present disclosure. Merely by way of example, the collimator 220 may be a pinhole collimator including pinholes as collimating units. In some embodiments, an aperture size of the pinholes may relate to the resolution and sensitivity of the imaging apparatus 200. For example, pinholes with a smaller aperture size (e.g., the second collimating unit having the second aperture size) may improve the resolution, and pinholes with a larger aperture size (e.g., the first collimating unit having the first aperture size) may improve the sensitivity. Therefore, to improve a trade-off between the resolution and the sensitivity, a hybrid collimator including the multiple types of collimating units (e.g., pinholes with different aperture sizes) in different configurations may be designed.

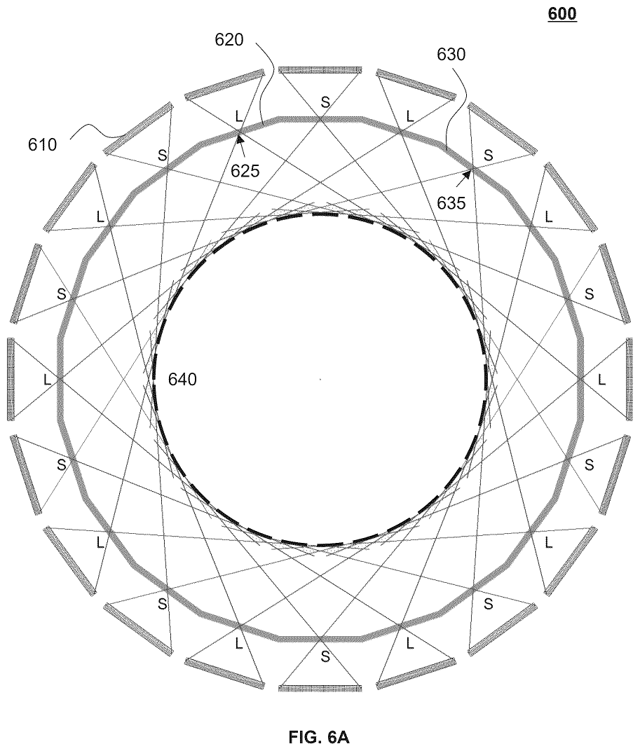

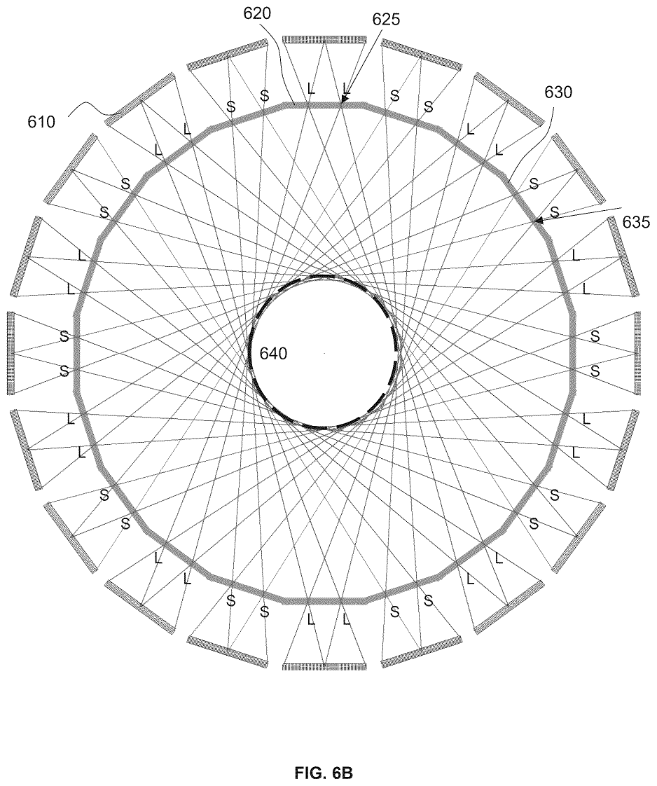

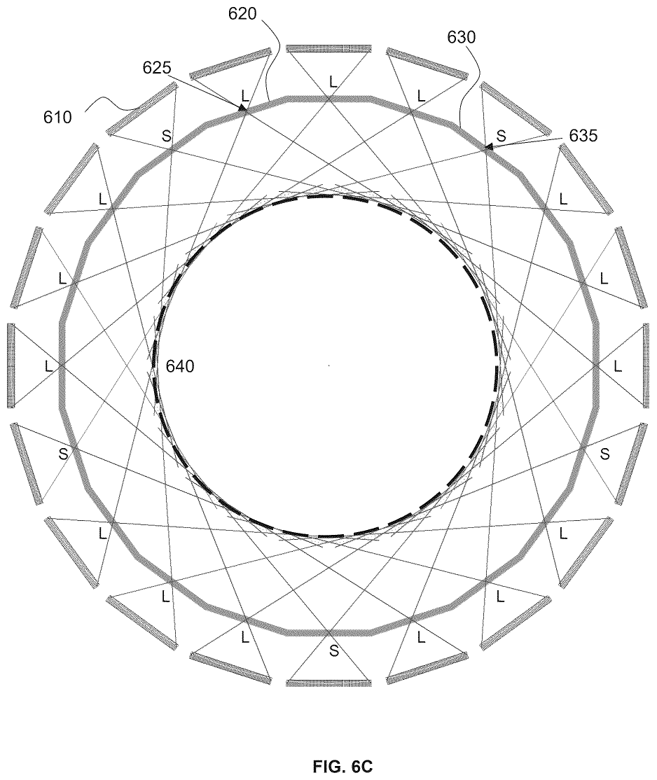

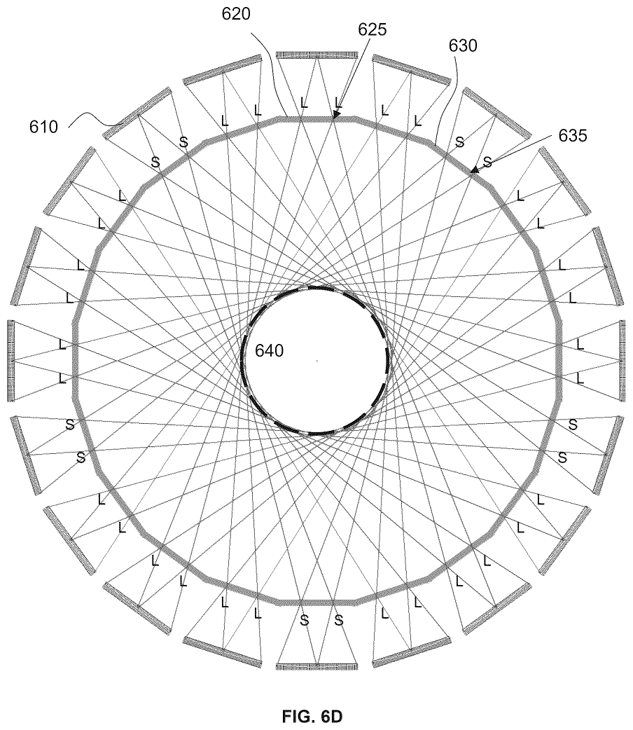

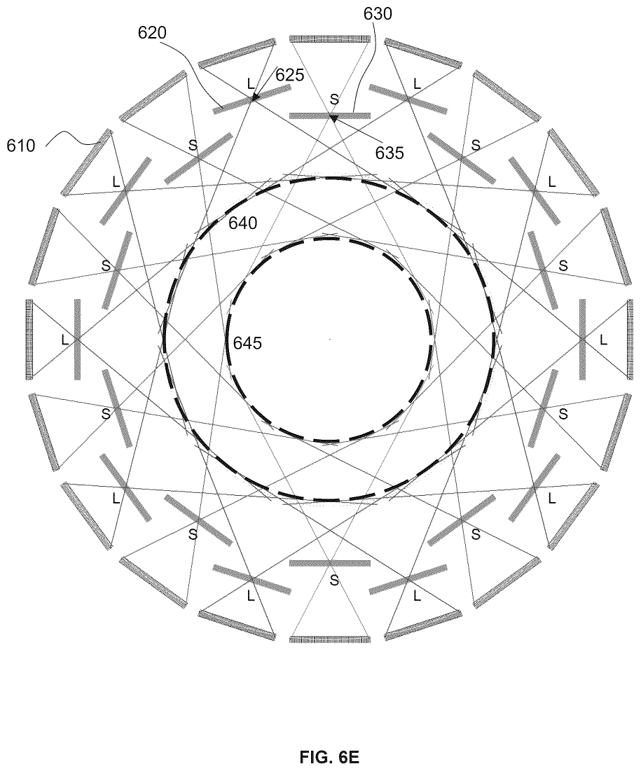

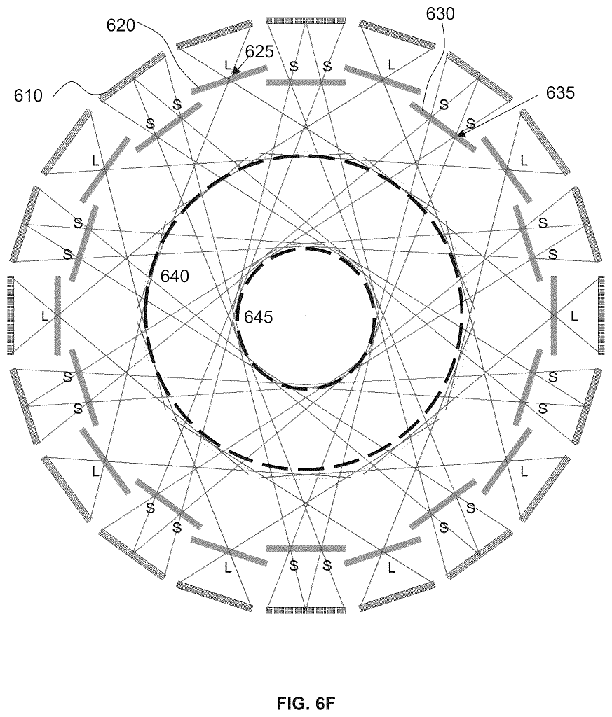

For example, referring to FIG. 6A, FIG. 6A is a schematic diagram illustrating a side view of an exemplary imaging apparatus 600 from an axial direction according to some embodiments of the present disclosure. The imaging apparatus 600 may include a detector including detecting modules 610 arranged along a circumference direction of the imaging apparatus 600 and a collimator including collimating modules arranged within the accommodation space along the circumference direction. The collimating modules may include collimating units. During the scan of the target subject, at least a portion of the collimating units are in an effective state for allowing radiation rays from the target subject to pass through. The at least a portion of the collimating units may include multiple types of collimating units in different configurations.

As shown in FIG. 6A, the multiple types of collimating units may include a first collimating unit 625 and a second collimating unit 635. The first collimating unit 625 may have a first aperture size (indicated as “L”) and be arranged on a first collimating module 620, and the second collimating unit 635 may have a second aperture size (indicated as “S”) smaller than the first aperture size and be arranged on a second collimating module 630 different from the first collimating module 620. The first collimating unit 625 may be also referred to as a large pinhole, and the second collimating unit 635 may be also referred to as a small pinhole. The large pinholes and the small pinholes may be alternately arranged along the circumference direction of the imaging apparatus 600. A first distance between the first collimating module 620 and a central axis of the imaging apparatus 600 may be the same as a second distance between the second collimating module 630 and the central axis of the imaging apparatus 600, and the scan may be a single-FOV scan. For instance, a dotted circle may indicate the single FOV 640 formed the first collimating unit 625 and the second collimating unit 635.

In some embodiments, by adjusting the configurations, the counts, the position, etc., of the collimating units in the effective state, the imaging apparatus including the hybrid collimator may be designed to satisfy the imaging requirement defined by one or more imaging parameters, such as the FOV, the sensitivity, the resolution, the detecting rate, etc.

For example, by modifying a count of collimating units in the effective state in each collimating module, the FOV 640 of the imaging apparatus 600 may be adjusted. Merely by way of example, as shown in FIG. 6A, each collimating module may include one collimating unit in the effective state during the scan, and the FOV 640 of the imaging apparatus 600 in FIG. 6A may be relatively large and correspond to a whole-body mode. As shown in FIG. 6B, each collimating module in FIG. 6B may include two collimating units in the effective state during the scan, and the FOV 640 of the imaging apparatus 600 in FIG. 6B may be relatively small and correspond to a focused FOV mode. As another example, as shown in FIGS. 6E and 6F, each second collimating module 630 in FIG. 6E may include one collimating unit 635 in the effective state during the scan, each second collimating module 630 in FIG. 6F may include two collimating units 635 in the effective state during the scan, and a distance between a dotted circle indicating an FOV 640 (also referred to as a first FOV) and a dotted circle indicating an FOV 645 (also referred to as a second FOV) in FIG. 6E may be respectively smaller than a distance between a dotted circle indicating an FOV 640 and a dotted circle indicating an FOV 645 in FIG. 6F. That is, the FOV 645 in FIG. 6E may have a larger area than the FOV 645 in FIG. 6F.

As another example, by modifying a ratio of a first count of the first collimating unit 625 to a second count of the second collimating unit 635, the resolution and the sensitivity of the imaging apparatus 600 may be adjusted. For instance, as shown in FIGS. 6A and 6C, by modifying the ratio of the first count of the first collimating unit 625 to the second count of the second collimating unit 635 from 1:1 to 3:1, the sensitivity of the imaging apparatus 600 may be improved, while the resolution of the imaging apparatus 600 may be reduced. Similarly, as shown in FIGS. 6B and 6D, by modifying the ratio of the first count of the first collimating unit 625 to the second count of the second collimating unit 635 from 1:1 to 3:1, the sensitivity of the imaging apparatus 600 may be improved, while the resolution of the imaging apparatus 600 may be reduced.

As still another example, by modifying the first distance to be different from the second distance, the single FOV may be adjusted to a multi-FOV (e.g., a dual FOV). For instance, as shown in FIGS. 6E and 6F, the first distance between the first collimating module 620 and the central axis of the imaging apparatus 600 may be different from the second distance between the second collimating module 630 and the central axis of the imaging apparatus 600, and the scan may be a dual-FOV scan. Further, since the first distance is greater than the second distance, the FOV 640 corresponding to the first collimating modules 620 (i.e., formed by the first collimating unit 625) may be located between the FOV 645 corresponding to the second collimating modules 630 (i.e., formed by the second collimating unit 635) and the collimating modules or the detecting modules 610.

In some embodiments, the dual FOV may be used to scan a target subject including a region of interest (ROI). For example, the FOV 640 corresponding to the first collimating modules 620 may cover the target subject, and the FOV 645 corresponding to the second collimating modules 630 may cover the ROI. For instance, in a cardiac scan, the first collimating modules 620 corresponding to the FOV 640 may be used to acquire image data corresponding to the chest, and the second collimating modules 630 corresponding to the FOV 645 may be used to acquire image data corresponding to the heart. Since the second collimating units in the second collimating modules have higher spatial resolution than the first collimating units in the first collimating modules, the imaging resolution and quality of the heart can be improved.

As yet another example, by modifying the first distance and/or the second distance, the FOV 640 and/or the FOV 645 of the imaging apparatus 600 may be adjusted. For instance, the larger the first distance (or the second distance), the larger the area of the FOV 640 (or the FOV 645).

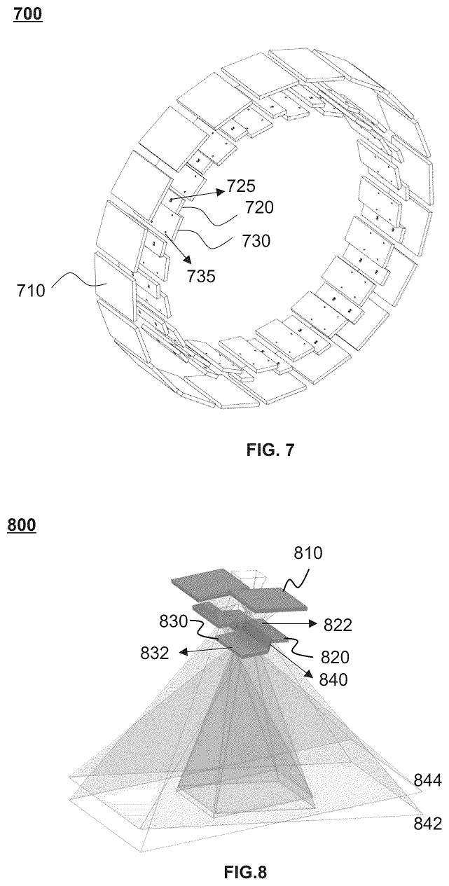

It should be noted that the collimating units in FIGS. 6A-6F are merely for illustration and not to limit the scope of the present disclosure. For example, each collimating module in FIG. 6B may include the first collimating unit 625 and the second collimating unit 635, the first distance between the first collimating unit 625 and the central axis of the imaging apparatus 600 may be the same as the second distance between the second collimating unit 635 and the central axis of the imaging apparatus 600, and the scan may be a single-FOV scan. Alternatively, the first distance between the first collimating unit 625 and the central axis of the imaging apparatus 600 may be different from the second distance between the second collimating unit 635 and the central axis of the imaging apparatus 600, and the scan may be a dual-FOV scan. As another example, since FIGS. 6A-6F are side views of the imaging apparatus 600, each single collimating unit shown in FIGS. 6A-6F may represent a row of collimating unit(s), and the row of collimating unit(s) may include multiple collimating units. Referring to FIGS. 7 and 8, a single collimating unit seen from the axial direction of an imaging apparatus actually represents a row of collimating units that includes two collimating units. In some embodiments, one row of collimating unit(s) may be regarded as a single collimating unit.

In some embodiments, a collimating module may include multiple collimating units with the same configuration, and different collimating modules may include collimating units with different configurations. That is, the structure parameters (e.g., an aperture size, a hole spacing, a hole channel length, an opening angle, etc.) of the collimating units included in the same collimating module may be the same, and the structure parameters of the collimating units in different collimating modules may be different. For example, as shown in FIGS. 6A-6F, the first collimating units 625 in the first collimating modules 620 may have the same configuration, and the second collimating units 635 in the second collimating modules 630 may have the same configuration. For instance, each of the first collimating units 625 may have a first aperture size, and each of the second collimating units 635 may have a second aperture size. As another example, referring to FIG. 7, FIG. 7 is a schematic diagram illustrating an exemplary imaging apparatus 700 according to some embodiments of the present disclosure. As shown in FIG. 7, the imaging apparatus 700 may include detecting modules 710, first collimating modules 720, and second collimating modules 730. First collimating units 725 in the first collimating modules 720 may have the same configuration, and second collimating units 735 in the second collimating modules 730 may have the same configuration.

In some embodiments, the structure parameters of the pinholes included in the same collimating unit may be different, that is, one collimating module may include multiple types of collimating units in different configurations.

In some embodiments, a collimating module and an adjacent collimating module may be connected through a connection member. The connection member may include a detachable connection, such as a clamping connection, a screw connection, a rivet connection, a hinge connection, or the like, or any combination thereof. For example, referring to FIG. 8, FIG. 8 is a schematic diagram illustrating an exemplary imaging apparatus 800 according to some embodiments of the present disclosure. As shown in FIG. 8, the imaging apparatus 800 may include detecting modules 810, first collimating modules 820, and second collimating modules 830. Each of the first collimating modules 820 may include first collimating units 822 each of which has a first aperture size, and each of the second collimating modules 830 may include second collimating units 832 each of which has a second aperture size smaller than the first aperture size. Each first collimating module 820 and an adjacent second collimating module 830 may be connected through a connection member 840. In some embodiments, a material of the connection member may be the same as or different from the material(s) of the collimating modules.

It should be noted that the collimators and/or the imaging apparatus in FIGS. 6A-8 are merely provided for illustration purposes, and not limit the scope of the present disclosure. For example, the configurations of the collimating units may be modified according to actual needs. For instance, the counts and the positions of the large pinholes and the small pinholes that are used (i.e., in the effective state) can be modified. As another example, the counts and the positions of the first collimating modules and the second collimating modules may be modified. As a further example, the collimator may further include one or more third collimating modules, and each of the third collimating modules may include at least one third collimating unit which has a third aperture size. The third aperture size may be different from the first aperture size and the second aperture size. For example, the third aperture size may be larger than the second aperture size and smaller than the first aperture size, and the third collimating unit may be also referred to as a medium pinhole.

In some embodiments, structure parameters of the collimator 220 may be determined before the scan of the target subject. For example, a desired resolution and a desired sensitivity of the imaging apparatus 200 may be determined based on information relating to the target subject (e.g., a body part to be scanned, scanning requirements) and/or a treatment plan, and reference values of the structure parameters of the collimator 220 (e.g., the structure parameters of the collimating units) may be based on the desired resolution and the desired sensitivity (e.g., according to below equations). Then, target values of the structure parameters of the collimator 220 may be determined based on the reference values of the structure parameters, for example, by performing data simulation. In some embodiments, the processing device 140 may obtain the scan data collected by the detector 210 by performing the scan on the target subject based on the target values of the structure parameters of the collimator 220 (or the confirmed or updated target values), and generate a target reconstruction image of the target subject by reconstructing the scan data. More descriptions regarding the generation of the target reconstruction image may be found elsewhere in the present disclosure. See, e.g., FIGS. 10-13, 18A, and 18B, and relevant descriptions thereof.

By arranging the collimator with the multiple types of collimating units in different configurations, the hybrid collimator can be designed to collect multi-scale scan data (e.g., projection data) in a single scan, which can leverage the advantages of small pinholes for improved resolution and large pinholes for increased sensitivity, thereby improving trade-offs among the resolution, the sensitivity, and the FOV of the imaging apparatus. Furthermore, by modifying the first distance and/or the second distance, scan data corresponding to the multi-FOV can be collected, which can increase the richness of imaging information of the target subject, thereby providing optimal imaging performance of the imaging apparatus and improving the quality of the image generated based on the scan data.

It should be noted that the foregoing descriptions are merely provided for the purpose of illustration and are not intended to limit the scope of the present disclosure. For those skilled in the art, amendments and variations may be made under the teaching of the descriptions of the present disclosure. However, these amendments and variations do not depart from the scope of the present disclosure.

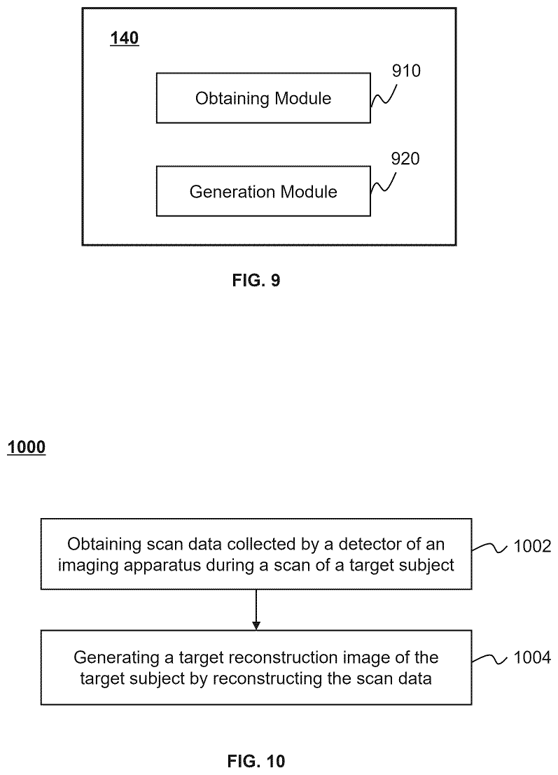

FIG. 9 is a block diagram illustrating an exemplary processing device 140 according to some embodiments of the present disclosure. In some embodiments, the processing device 140 may be in communication with a computer-readable storage medium (e.g., the storage device 150 illustrated in FIG. 1) and execute instructions stored in the computer-readable storage medium. The processing device 140 may include an obtaining module 910 and a generation module 920.

The obtaining module 910 may be configured to obtain scan data collected by a detector of an imaging apparatus during a scan of a target subject. The scan data may be collected by the detector by detecting radiation rays from the target subject that pass through at least a portion of collimating units of a collimator of the imaging apparatus, and the at least a portion of the collimating units may include multiple types of collimating units in different configurations. More descriptions regarding the obtaining of the scan data may be found elsewhere in the present disclosure. See, e.g., operation 1002 and relevant descriptions thereof.

The generation module 920 may be configured to generate a target reconstruction image of the target subject by reconstructing the scan data. More descriptions regarding the generation of the target reconstruction image may be found elsewhere in the present disclosure. See, e.g., operation 1004 and relevant descriptions thereof.

It should be noted that the above description regarding the processing device 140 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure. For example, the processing device 140 may include a storage module configured to store data generated by the above-mentioned modules of the processing device 140. As another example, the processing device 140 may further include a training module configured to train one or more machine learning models disclosed herein. As still another example, one or more modules may be integrated into a single module to perform the functions thereof.

FIG. 10 is a flowchart illustrating an exemplary process 1000 for generating a target reconstruction image according to some embodiments of the present disclosure. The process 1000 may be implemented in the imaging system 100 illustrated in FIG. 1. For example, the process 1000 may be stored in the storage device 150 in the form of instructions (e.g., an application), and invoked and/or executed by the processing device 140.

In 1002, the processing device 140 (e.g., the obtaining module 910) may obtain scan data collected by a detector of an imaging apparatus during a scan of a target subject.

The detector may include detecting modules arranged along a circumference direction of the imaging apparatus, and each detecting module may correspond to one or more collimating units of a collimator of the imaging apparatus in an effective state. For example, during the scan of the target subject, at least a portion of the collimating units of the collimator may be in the effective state for allowing radiation rays from the target subject to pass through and irradiate the detecting modules, and the at least a portion of the collimating units may include multiple types of collimating units in different configurations. More descriptions regarding the imaging apparatus and components thereof may be found elsewhere in the present disclosure. See, e.g., FIGS. 1-8 and relevant descriptions thereof.

The scan data refers to projection data of the radiation rays collected by the detector. In some embodiments, the imaging apparatus is a SPECT scanner, and the scan data includes SPECT data collected by the detector. In some embodiments, the scan data may be collected by the detector by detecting the radiation rays from the target subject that pass through the at least a portion of collimating units of the collimator of the imaging apparatus. For example, after a radioactive tracer is injected into the target subject, the radioactive tracer may decay to generate radiation rays (e.g., gamma rays). The gamma rays may be detected by the detector and converted into electrical signals by the detector. The electrical signals may be further converted into digit signals to be stored.

In some embodiments, the scan data may include information regarding radiation events (e.g., gamma photons) detected by the detector, such as, a count of the radiation events, an energy of each of the radiation events, an angle of each of the radiation events, a time when each of the radiation events is received, etc. In some embodiments, the scan data may be represented as sinograms which contain projection data from different angles.

In some embodiments, the scan data may include multiple data subsets corresponding to the multiple types of collimating units. For example, the multiple types of collimating units may include a first collimating unit (e.g., a large pinhole) and a second collimating unit (e.g., a small pinhole). Correspondingly, the scan data may include first scan data (also referred to as a first data subset) corresponding to the first collimating unit and second scan data (also referred to as a second data subset) corresponding to the second collimating unit. The first scan data refers to scan data collected by the detector after the radiation rays from the target subject pass through the first collimating units and irradiate the detecting modules, and the second scan data refers to scan data collected by the detector after the radiation rays from the target subject pass through the second collimating units and irradiate the detecting modules. For example, the processing device 140 may divide the scan data collected by a specific collimating module into the first scan data or the second scan data based on the type of collimating units that corresponds to the specific collimating module.

In some embodiments, during the scan of the target subject, the processing device 140 may cause the collimator to rotate relative to the detector around a central axis of the imaging apparatus by a preset step size one or more times. In such cases, the collimator has different relative positions with respect to the detector during the scan. Correspondingly, the scan data may include multiple scan data sets corresponding to the different relative positions.

In some embodiments, the preset step size may be determined such that each time the collimator rotates, projections of the at least a portion of the collimating units on the detecting modules are shifted by a distance smaller than a bin size of the detector. For example, the shift of the projections may be smaller than the bin size of the detector, and can be determined according to Equation (1):

$${S}_{step} = {kS}_{b i n} , \qquad ( 1 )$$

where Sₛₜₑₚ refers to the shift of the projections corresponding to the preset step size, S_{bin} refers to a bin size of the detector, and k refers to a coefficient. k is a value larger than 0 and smaller than 1, such as, 0.1, 0.2, 0.25, 0.3, 0.5, 0.75, 0.8, 0.9, etc. In such cases, each time the collimator rotates, projections of the at least a portion of the collimating unit on the detecting modules are shifted by a fraction of the bin size (e.g., half of the bin size).

In some embodiments, the less the Sₛₜₑₚ or k is, the longer scanning time the scan may last for, and the higher the resolution may be.

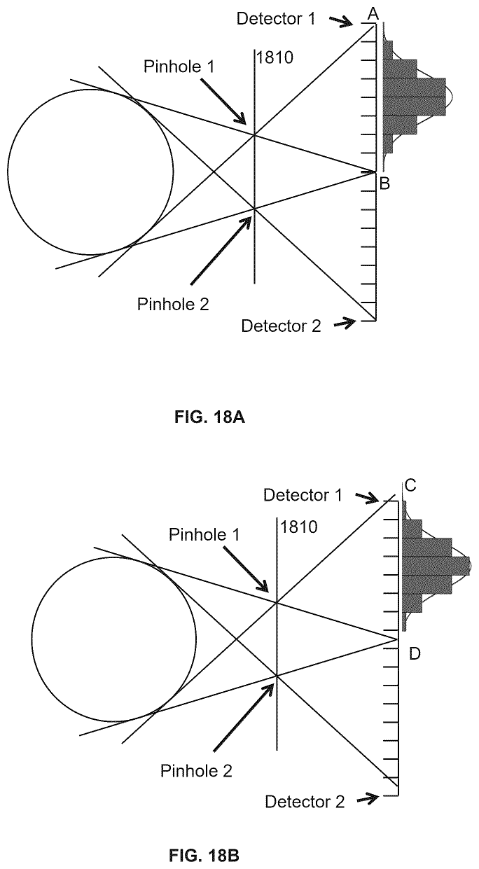

By rotating the collimator in finer steps, the relative movement between the detector and the collimator can be adjusted in small increments, such as, each fractionally smaller than the bin size of the detector as shown in FIGS. 18A and 18B. This can effectively simulate acquiring the scan data (e.g., the projection data) at finer spatial intervals than the inherent bin size of the detector, as each incremental movement captures the scan data at slightly different offsets, thereby acquiring the scan data with greater detail and precision and improving the imaging resolution, which is equivalent to acquiring the scan data at a higher resolution or using a smaller bin size.

In some embodiments, the processing device 140 may obtain the scan data from the imaging apparatus (e.g., the imaging apparatus 110, the imaging apparatus 200, the imaging apparatus 300, the imaging apparatus 600, the imaging apparatus 700, the imaging apparatus 800, etc.) or a storage device (e.g., the storage device 150, a database, or an external storage).

In 1004, the processing device 140 (e.g., the generation module 920) may generate a target reconstruction image of the target subject by reconstructing the scan data.

The target reconstruction image refers to a reconstruction image for presenting and/or analysis. For example, the target reconstruction image may include image information regarding the target subject (or an ROI of the target subject) for presenting to a user (e.g., a doctor, a technician, etc.).

In some embodiments, the processing device 140 may generate the target reconstruction image of the target subject by reconstructing the scan data through a reconstruction algorithm. Exemplary reconstruction algorithms may include an analytical algorithm (e.g., a filtered back-projection algorithm), an iteration algorithm, a maximum likelihood expectation-maximization (MLEM) algorithm, an ordered subset expectation maximization (OSEM) algorithm, a least squares (LS) algorithm, or the like, or any combination thereof.

In some embodiments, the target reconstruction image may be a multi-scale image. For example, when the first scan data and the second scan data correspond to different FOVs, the target reconstruction image may be the multi-scale image. Merely by way of example, referring to FIG. 6E and FIG. 6F, the scan on the target subject may be a dual-FOV scan having a first FOV 640 formed by the first collimating units 625 and a second FOV 645 formed by the second collimating units 635. Further, the target reconstruction image may include a second portion corresponding to the second FOV and a first portion other than the second portion, and the voxel size of voxels in the second portion may be smaller than the voxel size of voxels in the first portion. For example, if the target subject includes an ROI, the first FOV may cover the target subject, and the second FOV may cover the ROI; the second portion in the target reconstruction image may represent the ROI, the first portion in the target reconstruction image may represent other parts of the target subject, and the second portion may have smaller voxel size than the first portion.

Since a size of voxels relates to a resolution (e.g., a spatial resolution) of an image, the second portion of the target reconstruction image can have more detailed data and finer resolution than the first portion. Therefore, the resolution of the ROI can be improved, which can enhance a local recognition ability of the target reconstruction image.

In some embodiments, the processing device 140 may construct a three-dimensional (3D) voxel matrix for the target reconstruction image before the reconstruction. Each voxel in the 3D voxel matrix may have a certain voxel size, which indicates a size of a corresponding physical point in a physical space. For instance, the processing device 140 may determine the voxel size of the voxel based on a location of the corresponding physical point in the dual-FOV. Referring to FIG. 6E, if the corresponding physical point is located within the second FOV 645, the processing device 140 may determine a voxel size of the voxel as a small voxel size. If the corresponding physical point is located within the first FOV 640 and out of the second FOV 645, the processing device 140 may determine a voxel size of the voxel as a large voxel size. The small voxel size and the large voxel size may be determined based on a system default setting or set manually by a user.

In such cases, the target reconstruction image may be the multi-scale (multi-resolution) image, wherein a second portion corresponding to the second FOV has a higher resolution than the first portion other than the second portion.

In some embodiments, the processing device 140 may generate an initial reconstruction image of the target subject based on the first scan data, and generate the target reconstruction image of the target subject by updating the initial reconstruction image based on the second scan data. More descriptions regarding the generation of the target reconstruction image may be found elsewhere in the present disclosure. See, e.g., FIG. 11A and relevant descriptions thereof.

In some embodiments, the processing device 140 may generate the target reconstruction image of the target subject by reconstructing the scan data through an iterative process including iterations. Each iteration may include: for each type of collimating unit, generating an intermediate reconstruction image based on the data subset corresponding to the type of collimating unit and a combined reconstruction image of a previous iteration; generating a combined reconstruction image of the current iteration by combining the intermediate reconstruction images of the multiple types of collimating units based on weights of the multiple types of collimating units; and proceeding to a next iteration or designating the combined reconstruction image of the current iteration as the target reconstruction image. More descriptions regarding the generation of the target reconstruction image may be found elsewhere in the present disclosure. See, e.g., FIG. 11B and relevant descriptions thereof.

In some embodiments, before operation 1002 is performed, the processing device 140 may determine target values of structure parameters and counts of the multiple types of collimating units in the collimator by performing process 1300 in FIG. 13. Further, the processing device 140 may select a suitable collimator to be used during the scan from multiple available collimators based on the target values of the structure parameters and the counts, and output a message for reminding a user to assemble the selected collimator in the imaging apparatus. As another example, if the imaging apparatus has already assembled with a collimator or the collimator of the imaging apparatus is non-detachable, the processing device 140 may adjust the collimator by controlling it to rotate to a suitable position and/or enabling/disabling one or more shielding modules to achieve the target values of structure parameters and counts of the multiple types of collimating units. After the collimator is assembled or adjusted, the processing device 140 may control the imaging apparatus to scan the target subject.

According to some embodiments of the present disclosure, by introducing the specially designed structure of the hybrid collimator, the multi-scale scan data (i.e., the multiple data subsets corresponding to the multiple types of collimating units) can be obtained in a single scan, which can increase the richness of imaging information of the target subject, thereby improving trade-offs among the resolution, the sensitivity, and the FOV of the imaging apparatus.

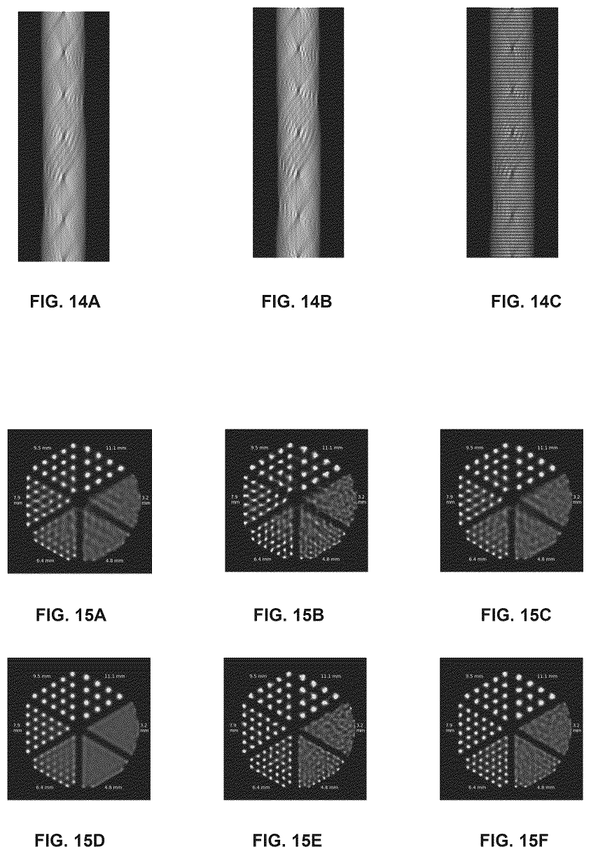

For example, as shown in FIGS. 14A-14C, an image (indicating data in sinogram format) in FIG. 14C has a higher image resolution than an image in FIG. 14A and more information than an image in FIG. 14B, wherein the image in FIG. 14A is obtained through large pinholes, the image in FIG. 14B is obtained through small pinholes, and the image in FIG. 14C is obtained through hybrid pinholes. As another example, as shown in FIGS. 15A-15C, points in a reconstruction image in FIG. 15A are circular but fuzzy (high sensitivity but low resolution), points in a reconstruction image in FIG. 15B are clear but irregular (high resolution but low sensitivity), and points in a reconstruction image in FIG. 15C are relatively circular and clear (relatively high resolution and sensitivity), wherein the reconstruction image in FIG. 15A is generated based on scan data (with a low count of radiation events) obtained through large pinholes, the reconstruction image in FIG. 15B is generated based on scan data (with a low count of radiation events) obtained through small pinholes, and the reconstruction image in FIG. 15C is generated based on scan data (with a low count of radiation events) obtained through hybrid pinholes. That is, the scan data which is collected through the hybrid pinholes (or the hybrid collimator) can improve the trade-off between the resolution and the sensitivity. As still another example, as shown in FIGS. 15D-15F, points in a reconstruction image in FIG. 15D are circular but fuzzy (high sensitivity but low resolution), points in a reconstruction image in FIG. 15E are clear but irregular (high resolution but low sensitivity), and points in a reconstruction image in FIG. 15F are relatively circular and clear (relatively high resolution and sensitivity), wherein the reconstruction image in FIG. 15D is generated based on scan data (with a high count of radiation events) obtained through large pinholes, the reconstruction image in FIG. 15E is generated based on scan data (with a high count of radiation events) obtained through small pinholes, and the reconstruction image in FIG. 15F is generated based on scan data (with a high count of radiation events) obtained through hybrid pinholes.

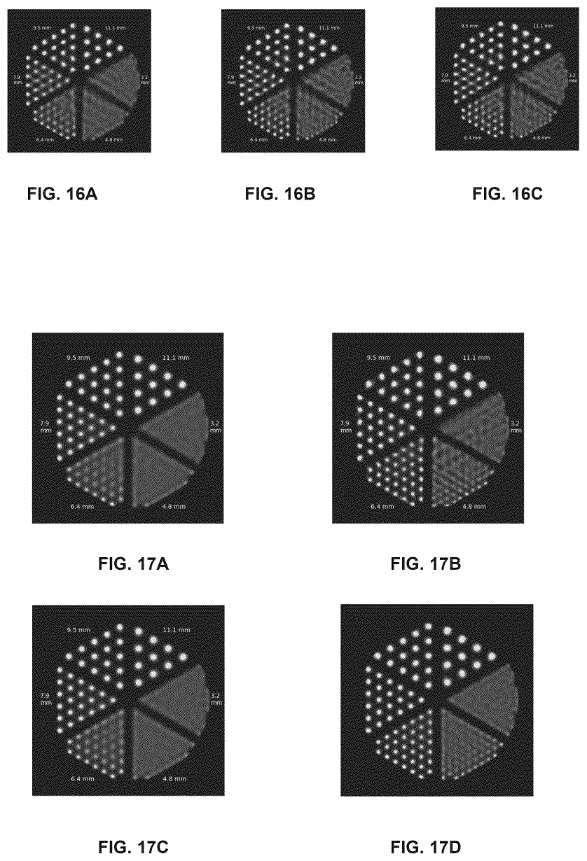

In addition, the trade-off between the resolution and the sensitivity can be adjusted based on a ratio of a first count of the first collimating units to a second count of the second collimating units. For example, as shown in 16A-16C, points in a reconstruction image in FIG. 16A are circular but fuzzy (high sensitivity but low resolution), points in a reconstruction image in FIG. 16B are relatively circular and clear (relatively high resolution but low sensitivity), and points in a reconstruction image in FIG. 16C are clear but irregular (high resolution but low sensitivity), wherein the reconstruction image in FIG. 16A is generated based on scan data obtained when the ratio is 3:1, the reconstruction image in FIG. 16B is generated based on scan data obtained when the ratio is 1:1, and the reconstruction image in FIG. 16C is generated based on scan data obtained when the ratio is 1:3.

Furthermore, in some embodiments, the target reconstruction image can be generated based on the multiple data subsets corresponding to different FOVs (or aperture sizes), which can include global information of the target subject corresponding to the large FOV (or large aperture size) and local information with a high resolution corresponding to the small FOV (or small aperture size), thereby improving the imaging performance of the imaging apparatus.

FIG. 11A is a schematic diagram illustrating an exemplary process 1100 for generating a target reconstruction image according to some embodiments of the present disclosure. In some embodiments, the process 1100 may be performed to achieve at least part of operation 1004 as described in connection with FIG. 10.

As shown in FIG. 11A, the processing device 140 may obtain scan data 1110 of a target subject. The scan data 1110 may include first scan data 1112 corresponding to the first collimating unit and second scan data 1114 corresponding to the second collimating unit, wherein the first collimating unit has a larger aperture size than the second collimating unit. The processing device 140 may generate an initial reconstruction image 1120 of the target subject based on the first scan data 1112, and generate a target reconstruction image 1130 of the target subject by updating the initial reconstruction image 1120 based on the second scan data 1114.

The initial reconstruction image 1120 refers to an image with a low resolution. In some embodiments, the initial reconstruction image 1120 may correspond to a first FOV (e.g., the first FOV 640). The initial reconstruction image 1120 may be generated by reconstructing the scan data 1110 through a reconstruction algorithm. The reconstruction algorithm may be similar to the reconstruction algorithm described in FIG. 10.

The target reconstruction image 1130 refers to an image with improved resolution compared with the initial reconstruction image 1120. If the first collimating unit and the second collimating unit form different FOVs (e.g., as shown in FIGS. 6E and 6F), the initial reconstruction image 1120 may have a relatively large FOV, and the target reconstruction image 1130 may be a multi-scale image with multiresolution. For example, the target reconstruction image 1130 may include a second portion corresponding to a second FOV formed by the second collimating unit (e.g., the second FOV 645) and a first portion other than the second portion. The first portion may have a low resolution, and the second portion may have a high resolution.

In some embodiments, the processing device 140 may generate the target reconstruction image 1130 by iteratively updating the initial reconstruction image 1120 based on the second scan data 1114. For example, an iterative reconstruction process is performed based on the second scan data 1114 according to a reconstruction algorithm with the initial reconstruction image 1120 as the initial value of the iterative reconstruction process. In some embodiments, the processing device 140 may further determine correction data based on the initial reconstruction image 1120, and iteratively update the initial reconstruction image 1120 based on the second scan data 1114 and the correction data. The correction data may include an attenuation coefficient map of the target subject used for attenuation correction. For example, the processing device 140 may determine tissue types of different portions of the target subject (e.g., a target tissue type of an ROI in the second FOV) based on the initial reconstruction image 1120, and determine the attenuation coefficient map based on the tissue types. By updating the initial reconstruction image based on the second scan data and the correction data, the reconstruction efficiency of the second scan data and the accuracy of the target reconstruction image can be improved.

In some embodiments, the processing device 140 may generate a second initial reconstruction image based on the second scan data 1114, and generate the target reconstruction image by fusing the initial reconstruction image 1120 and the second initial reconstruction image. For example, the processing device 140 may determine weight values of the initial reconstruction image 1120 and the second initial reconstruction image, and fuse the two initial reconstruction images based on the weight values.

According to some embodiments of the present disclosure, the target reconstruction image can be generated based on the first scan data and the second scan data corresponding to different aperture sizes and/or different FOVs. The target reconstruction image can be the multi-scale image, which includes not only global information from the first scan data but also local information with a high resolution from the second scan data, thereby improving the imaging performance of the imaging apparatus.

FIG. 11B is a schematic diagram illustrating an exemplary process 1150 for generating a target reconstruction image according to some embodiments of the present disclosure. In some embodiments, the process 1150 may be performed to achieve at least part of operation 1004 as described in connection with FIG. 10.

As described above, multiple types of collimating units are in the effective state during the scan, and the scan data includes multiple data subsets corresponding to multiple collimating units. For example, as shown in FIG. 11B, multiple types of collimating units may include a first collimating unit and a second collimating unit, and scan data may include a data subset 1162 corresponding to the first collimating unit and a data subset 1164 corresponding to the second collimating unit. The processing device 140 may generate the target reconstruction image 1130 of a target subject by reconstructing the scan data through an iterative process including iterations.

For a current iteration, the processing device 140 may generate an intermediate reconstruction image corresponding to each type of collimating unit based on the data subset corresponding to the type of collimating unit and a combined reconstruction image of a previous iteration (also referred to as a previous combined reconstruction image), for example, according to Equation (7) below. In some embodiments, when the current iteration is the first iteration in the iteration process, a blank image may be designated as the previous combined reconstruction image. The blank image refers to an image whose pixel values are initial values, such as, 0. When the current iteration is an iteration other than the first iteration, the combined reconstruction image of the previous iteration may be designated as the previous combined reconstruction image. For example, as shown in FIG. 11B, the processing device 140 may generate an intermediate reconstruction image 1172 based on the data subset 1162 and a previous combined reconstruction image 1152, and generate an intermediate reconstruction image 1174 based on the data subset 1164 and the previous combined reconstruction image 1152.

Then, the processing device 140 may generate a combined reconstruction image of the current iteration (also referred to as a current combined reconstruction image) by combining the intermediate reconstruction images of the multiple types of collimating units based on weights of the multiple types of collimating units, for example, according to Equation (5) or (9). For example, as shown in FIG. 11B, the processing device 140 may generate a current combined reconstruction image 1182 by combining the intermediate reconstruction image 1172 and the intermediate reconstruction image 1174 based on a first weight corresponding to the first collimating unit and a second weight corresponding to the second collimating unit.

Further, the processing device 140 may determine whether the current combined reconstruction image 1182 satisfies a preset condition. The preset condition may include that a preset count of iterations has been performed, that a difference between the current combined reconstruction image and the previous combined reconstruction image is 1152 less than a preset threshold, etc. If the current combined reconstruction image 1182 satisfies the preset condition, the processing device 140 may designate the current combined reconstruction image 1182 as the target reconstruction image 1130; otherwise, the processing device 140 may proceed to a next iteration. In the next iteration, the current combined reconstruction image 1182 is designated as the previous combined reconstruction image 1152.

In some embodiments, the weight of a type of collimating unit may indicate an importance degree of the type of collimating unit. For example, the larger the weight of the type of collimating unit is, the higher the importance degree of the type of collimating unit may be, and the larger the influence of the scan subset corresponding to the type of collimating unit on the target reconstruction image may be. The weight may be preset by a user or be determined by the processing device 140.

In some embodiments, the weight of a type of collimating unit may be determined based on a sensitivity map of the type of collimating unit. For example, the first weight corresponding to the first collimating unit may be determined based on a sensitivity map of the first collimating unit, and the second weight corresponding to the second collimating unit may be determined based on a sensitivity map of the second collimating unit. The sensitivity map of the type of collimating unit may indicate probabilities that radiation events at different physical points in space can be detected by detecting modules corresponding to the type of collimating unit. If the probability at a specific physical point is relatively high, a corresponding value in the sensitivity map may be relatively large. At this time, the accuracy of the data corresponding to the physical point may be relatively high. In some embodiments, the sensitivity map may be determined by performing a back projection on the system matrix corresponding to the type of collimating unit. In some embodiments, the sensitivity map for the type of collimating unit may be determined by multiplying the transpose of the system matrix corresponding to the pinhole type

$$( i . e . , {H}_{p}^{T} )$$

with a vector of 1.

In some embodiments, the weight of the type of collimating unit may be determined based on a ratio of the sensitivity map of the type of collimating unit to a sum of the sensitivity maps of all types of collimating units in the collimator. For example, the ratio may be designated as the weight of the type of collimating unit, such as according to Equation (6). As another example, the weight of the type of collimating unit may be determined based on the ratio and other information. For instance, the weight of the type of collimating unit may be determined based on the ratio and a second ratio of a count of the type of collimating unit and a sum count of all types of collimating units in the collimator.

By determining the weight of the type of collimating unit based on the sensitivity map, the contributions from each type of collimating unit can be appropriately weighted, leveraging their complementary properties to achieve high-quality image reconstruction.

In some embodiments, the weight of a type of collimating unit may be determined based on a scaling factor corresponding to the type of collimating unit. The scaling factor may be configured to adjust (e.g., scale) an initial weight of the type of collimating unit. For example, the scaling factor may be multiplied with an initial weight, which is determined based on a sensitivity map of the type of collimating unit or a preset weight of the type of collimating unit. In some embodiments, the scaling factor may be associated with the count of radiation events detected by a detector in a scan of the target subject. For example, when the count of radiation events is relatively large (e.g., greater than a first threshold), the second weight corresponding to the second collimating unit may be improved, so as to improve the resolution of the image apparatus. That is, the scaling factor corresponding to the second collimating unit may be designated as a value larger than 1. As another example, when the count of radiation events is relatively small (e.g., smaller than a second threshold), the first weight corresponding to the first collimating unit may be improved, so as to reduce the noise in the scan data and improve a signal-to-noise ratio (SNR), thereby improving the imaging quality. That is, the scaling factor corresponding to the first collimating unit may be designated as a value larger than 1.

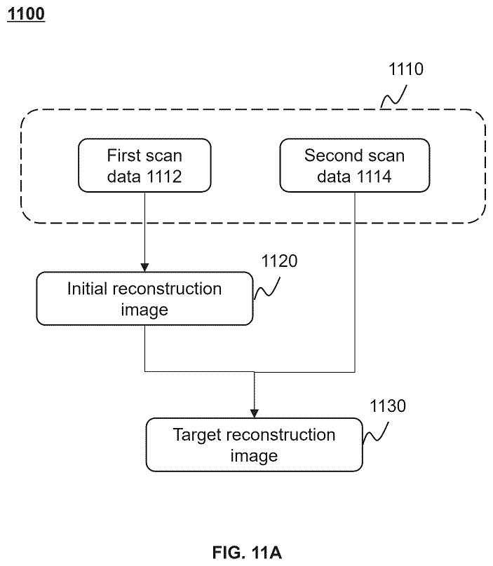

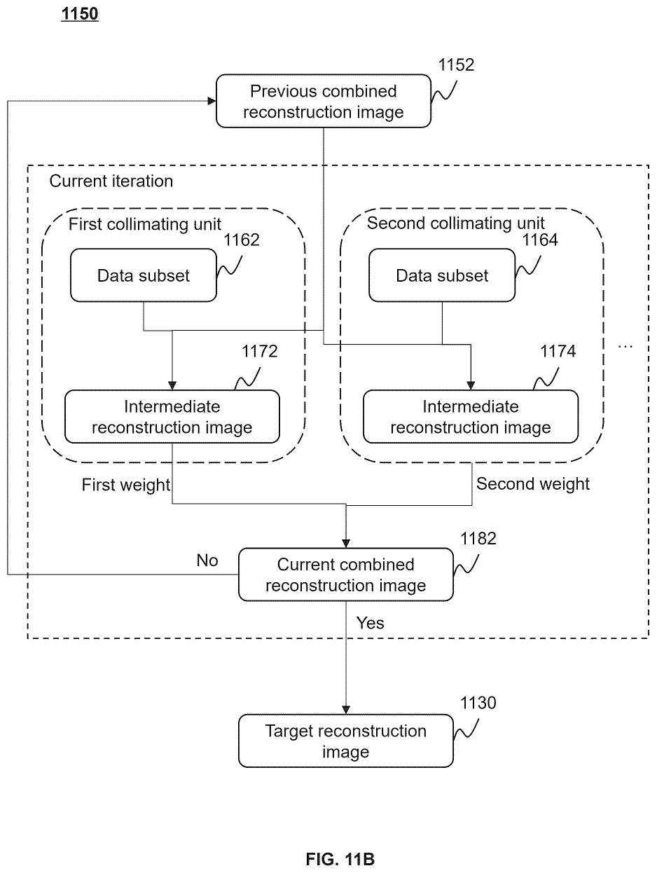

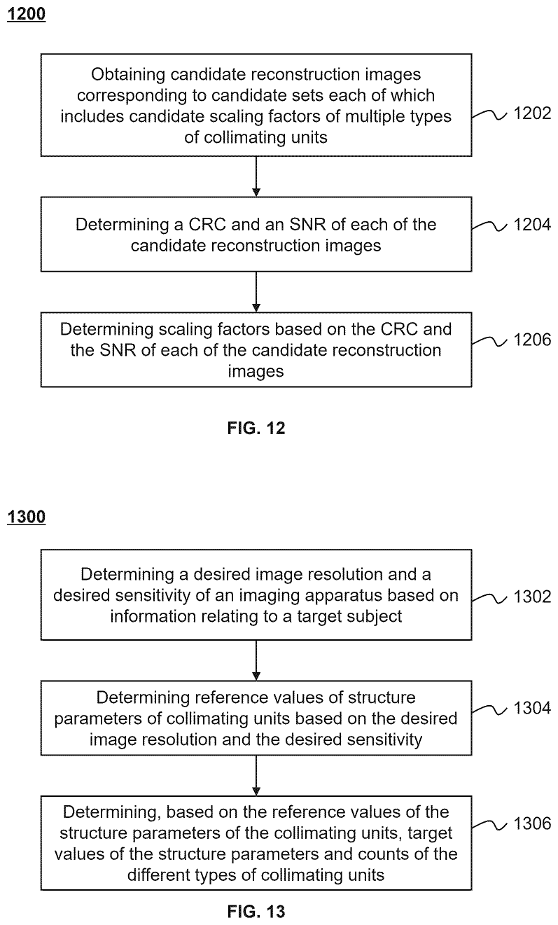

In some embodiments, the scaling factor may be determined based on candidate reconstruction images. For example, the processing device 140 may obtain candidate reconstruction images corresponding to candidate sets each of which includes candidate scaling factors of the multiple types of collimating units, and determine a contrast recovery coefficient (CRC) and a signal-to-noise ratio (SNR) of each of the candidate reconstruction images. Further, the processing device may determine the scaling factors based on the CRC and the SNR of each of the candidate reconstruction images. More descriptions regarding the determination of the scaling factors may be found elsewhere in the present disclosure. See, e.g., FIG. 12 and relevant descriptions thereof.

In some embodiments, the scaling factor may be determined based on other reference information, for example, scanning requirements, information relating to the target subject (e.g., a type, a size, a location of the body part to be scanned), etc.

By determining the weight of the multiple types of collimator units based on the CRC and SNR of the candidate reconstructed images, the contribution of the multiple types of collimator units can be optimized according to the imaging quality, thereby improving the overall imaging quality and imaging efficiency.

For illustration purposes, the reconstruction of scan data collected by an imaging apparatus including large pinholes, medium pinholes, and small pinholes is taken as an example and described below. The scan data may be also referred to as hybrid-pinhole data. A reconstruction algorithm may be used to reconstruct the scan data. Since the reconstruction algorithm plays a vital role in jointly reconstructing high-quality images from the hybrid-pinhole data, a specialized reconstruction approach based on Maximum Likelihood Expectation Maximization (MLEM) and Ordered Subset Expectation Maximization (OSEM) may be introduced to accurately account for Poisson statistics. By modeling the probability of observing the hybrid-pinhole data as a Poisson distribution, a likelihood function for parameters corresponding to the high-resolution image to be reconstructed may be designed. Using an Expectation Maximization (EM) strategy, the high-resolution image may be iteratively estimated by first calculating the expected log-likelihood in an expectation step, and then maximizing this log-likelihood in a maximization step to derive an update equation that progressively improves the reconstruction with each iteration. The expectation of the acquisition hybrid-pinhole data gₚ may be related to the target subject through Equation (2). That is, a relationship between the target reconstruction image of the target subject and the scan data may be represented as Equation (2):

$${\overline{g}}_{p} = {H}_{p} f + {r}_{p} , ( p = S , M , L ) \qquad ( 2 )$$

where p refers to a type of pinholes (e.g., the large pinholes, the medium pinhole, and the small pinholes) (also referred to as a pinhole type), L refers to the large pinholes, M refers to the medium pinhole, S refers to the small pinholes, g̅ₚ refers to the data subset corresponding to the pinhole type p, Hₚ refers to a tomographic forward projector or system matrix of the pinhole type p, rₚ refers to expected background events (e.g., random and scattered events) of the pinhole type p, and f refers to an activity distribution map/image in the target subject to be reconstructed (i.e., the target reconstruction image). Hₚ and rₚ may be determined by data simulation.

The processing device 140 may rearrange Equation (2) into a matrix form, so as to obtain Equation (3):

$$[ \begin{matrix}{\overline{g}}_{S} \\ {\overline{g}}_{M} \\ {\overline{g}}_{L}\end{matrix} ] = [ \begin{matrix}{H}_{S} \\ {H}_{M} \\ {H}_{L}\end{matrix} ] f + [ \begin{matrix}{r}_{S} \\ {r}_{M} \\ {r}_{L}\end{matrix} ] . \qquad ( 3 )$$

Equation (3) may represent a system model for scan data acquisition and system matrix. Correspondingly, the iterative image reconstruction is performed for the hybrid pinhole according to Equation (4):

$${\hat{f}}^{( k + 1 )} = \frac{{\hat{f}}^{( k )}}{{\sum }_{p = SM , L} {H}_{p}^{T} 1} {\sum }_{p = S , M , L} {H}_{p}^{T} \frac{{g}_{p}}{{H}_{p} {\hat{f}}^{( k )} + {r}_{p}} , \qquad ( 4 )$$

where

$${H}_{p}^{T} 1$$

refers to a sensitivity map for the pinhole type p, k refers to a count of iterations, \hat{f}^{(k)} refers to a reconstruction image in the k^{th} iteration, and \hat{f}^{(k+1)} refers to a reconstruction image in the (k+1)^{th} iteration. Assuming that the k^{th} iteration is the previous iteration, \hat{f}^{(k+1)} can be regarded as the combined reconstruction image of the current iteration as described above, \hat{f}(k) can be regarded as the combined reconstruction image of the previous iteration as described above.

The equation (4) iteratively refines the image reconstruction by balancing contributions from different pinhole types, incorporating sensitivity maps as weights of different pinhole types, to produce high-quality tomographic images.

Equation (4) provides a framework for iterative image reconstruction in a hybrid pinhole system, integrating contributions from different pinhole types to achieve optimal reconstruction. Equation (4) can be rewritten as Equation (5) below:

$${\hat{f}}^{( k + 1 )} = {\sum }_{p = S , M , L} {w}_{p} {\hat{f}}_{p}^{( k + 1 )} , \qquad ( 5 )$$

where wₚ refers to a weight for the pinhole type p, and

$${\hat{f}}_{p}^{( k + 1 )}$$

refers to an image update for the pinholes p in the (k+1)^{th} iteration, wherein wₚ and

$${\hat{f}}_{p}^{( k + 1 )}$$

are defined as Equations (6) and (7), respectively:

$${w}_{p} = \frac{{H}_{p}^{T} 1}{{\sum }_{p = S , M , L} {H}_{p}^{T} 1} , \qquad ( 6 )$$

$${\hat{f}}_{p}^{( k + 1 )} = \frac{{\hat{f}}^{( k )}}{{H}_{p}^{T} 1} {H}_{p}^{T} \frac{{g}_{p}}{{H}_{p} {\hat{f}}^{( k )} + {r}_{p}} . \qquad ( 7 )$$

Equation (6) may indicate that a weight of the pinhole type p is equal to a ratio of the sensitivity map of the pinhole type p to a sum of the sensitivity maps of all pinhole types.

$${\hat{f}}_{p}^{( k + 1 )}$$

can be regarded as the intermediate reconstruction image corresponding to a type of collimating unit in the current iteration as described above.

In some embodiments, to provide flexibility of the weights of different pinhole types, a scaling factor corresponding to the pinhole type p may be introduced to determine (e.g., reweight or update) the weight of the pinhole type p. For instance, the scaling factor corresponding to the pinhole type p may be added to Equation (6), so as to obtain Equation (8):

$${w}_{p}^{′} = \frac{{α}_{p} {H}_{p}^{T} 1}{{\sum }_{p = S , M , L} {α}_{p} {H}_{p}^{T} 1} = {α}_{p} \frac{{H}_{p}^{T} 1}{{\sum }_{p = S , M , L} {H}_{p}^{T} 1} , \qquad ( 8 )$$

where

$$\frac{{H}_{p}^{T} 1}{{\sum }_{p = S , M , L} {H}_{p}^{T} 1}$$

refers to an initial weight determined based on the sensitivity map of the pinhole type p, w′ₚ refers to the weight for the pinhole type p that has been reweighted or updated based on the scaling factor corresponding to the pinhole type p, and αₚ refers to a scaling factor corresponding to the pinhole type p.

Correspondingly, by substituting Equations (7) and (8) into Equation (5), the processing device 140 may obtain Equation (9):

$${\hat{f}}^{( k + 1 )} = \frac{{\hat{f}}^{( k )}}{{\sum }_{p = S , M , L} {α}_{p} {H}_{p}^{T} 1} {\sum }_{p = S , M , L} {α}_{p} {H}_{p}^{T} \frac{{g}_{p}}{{H}_{p} {\hat{f}}^{( k )} + {r}_{p}} . \qquad ( 9 )$$

In some embodiments, the target reconstruction image may be generated by iteratively reconstructing the scan data based on Equation (9). For example, the processing device 140 may determine whether \hat{f}^{(k+1)} satisfies the preset condition. If \hat{f}^{(k+1)} satisfies the preset condition, the processing device 140 may designate \hat{f}^{(k+1)} as the target reconstruction image; otherwise, the processing device 140 may proceed to a next iteration.

By introducing the weights of the multiple types of collimating units in the iterative reconstruction process, the contributions of different types of collimating units are balanced and appropriately weighted, leveraging their complementary properties to achieve high-quality image reconstruction. At the same time, multiple factors (e.g., the sensitivity map, the count of radiation events, etc.) are considered in the weight setting, which improves the accuracy of the weights and in turn, the image reconstruction accuracy.

For example, as shown in 17A-17D, points in a reconstruction image in FIG. 17A are circular but fuzzy (high sensitivity but low resolution), points in a reconstruction image in FIG. 17B are clear but irregular (high resolution but low sensitivity), points in a reconstruction image in FIG. 17C are relatively circular but fuzzy (relatively high resolution but relatively low sensitivity), and points in a reconstruction image in FIG. 17D are relatively circular and clear (relatively high resolution and sensitivity), wherein the reconstruction image in FIG. 17A is generated based on scan data obtained when a ratio of the first weight of the first collimating units to the second weight of the second collimating units is 1:0, the reconstruction image in FIG. 17B is generated based on scan data obtained when the ratio is 0:1, the reconstruction image in FIG. 17C is generated based on scan data obtained when the ratio is 1:1, and the reconstruction image in FIG. 17D is generated based on scan data obtained when the ratio is 4:1.

FIG. 12 is a flowchart illustrating an exemplary process 1200 for determining scaling factors according to some embodiments of the present disclosure. In some embodiments, the process 1200 may be performed to achieve at least part of the operations as described in connection with FIG. 11.

In 1202, the processing device 140 (e.g., the obtaining module 910) may obtain candidate reconstruction images corresponding to candidate sets each of which includes candidate scaling factors of multiple types of collimating units.

A candidate scaling factor refers to a candidate value for one type of collimating units. For example, the candidate scaling factors of a type of collimating unit may be random values within a particular range of values or user-specified values corresponding to the type of collimating unit. For instance, an initial scaling factor may be determined based on the count of radiation events detected by the detector in the scan of the target subject, and the candidate scaling factors may be determined from a range where the initial scaling factor is located.

A candidate reconstruction image corresponding to a candidate set refers to a reconstruction image generated based on the candidate set. In some embodiments, the candidate reconstruction image may be generated by performing image reconstruction on sample scan data of a sample subject (e.g., a phantom) based on the candidate scaling factors in the candidate set. The sample scan data may be obtained in a similar manner as how the scan data is obtained as described in operation 1002.

In some embodiments, the candidate reconstruction image may be generated in a similar manner as how the target reconstruction image is generated as described in operation 1004.

In some embodiments, the candidate reconstruction image may be generated by data simulation. For example, the Monte Carlo simulation technique is used to simulate the sample scan data of the sample subject, and the candidate reconstruction image may be generated based on the simulated scan data and the candidate scaling factors.

In some embodiments, the candidate reconstruction image may be an estimated image output by an image reconstruction model after information relating to the sample subject (e.g., an anatomical image), information relating to the scan of the sample subject (e.g., the scanned time, the tracer used in the scan), and the candidate scaling factors are input into the image reconstruction model. The image reconstruction model may be a trained machine learning model. In some embodiments, the image reconstruction model may be generated by a computing device (e.g., the processing device 140) by training an initial model using a plurality of training samples. Each of the plurality of training samples may include sample information of a sample subject, sample information relating to a sample scan of the sample subject, sample scaling factors, and a sample reconstruction image as a label. The training sample may be obtained from historical data.

In 1204, the processing device 140 (e.g., the generation module 920) may determine a CRC and an SNR of each of the candidate reconstruction images.

The CRC refers to an index that evaluates a recovery ability to the true contrast of a subject (e.g., a lesion or target) relative to its background. The SNR refers to an index that evaluates an image quality of the image by comparing the strength of the desired signal to the background noise.

In 1206, the processing device 140 (e.g., the generation module 920) may determine scaling factors based on the CRC and the SNR of each of the candidate reconstruction images.

For each candidate set, the processing device 140 may determine a value of a loss function based on the CRC of the candidate reconstruction image corresponding to the candidate set and the SNR of the candidate reconstruction image corresponding to the candidate set. The processing device 140 may select a target set from the candidate sets based on the value of the loss function corresponding to each candidate set, and designate the candidate scaling factors in the target set as the scaling factors.

The loss function may reflect a relationship between the CRC and the SNR of a candidate reconstruction image. For example, the loss function may measure a ratio of the CRC to the SNR. The candidate set corresponding to the largest value of the cost function may be determined as the target set, and the candidate scaling factors in the target set may be designated as the scaling factors.

As another example, the loss function may measure a first deviation of the CRC from a desired CRC and a second deviation of the SNR from a desired SNR (e.g., a sum or a weighted sum of the first deviation and the second deviation). The desired CRC and the desired SNR may be values that apply to the scan of the target subject. In some embodiments, the desired CRC and the desired SNR may be determined based on information relating to the target subject or set by a user. The candidate set corresponding to the smallest value of the cost function may be determined as the target set, and the candidate scaling factors in the target set may be designated as the scaling factors.

By determining the scaling factors based on the CRC and SNR of the candidate reconstructed images, the weights of multiple types of collimator units can be determined, and the contribution of the multiple types of collimator units can be optimized according to the imaging quality, thereby improving the overall imaging quality and imaging efficiency.

FIG. 13 is a flowchart illustrating an exemplary process 1300 for determining target values of structure parameters of a collimator according to some embodiments of the present disclosure.

In 1302, the processing device 140 (e.g., the generation module 920) may determine a desired resolution and a desired sensitivity of an imaging apparatus based on information relating to a target subject.

The information relating to the target subject may include a body part to be scanned, scanning requirements, a treatment plan, or the like, or any combination thereof.

The desired resolution refers to an optimal resolution for scanning the target subject.

A sensitivity refers to a probability that an emitted radiation signal is detected. The desired sensitivity refers to an optimal sensitivity for scanning the target subject.

In some embodiments, the processing device 140 may determine the desired resolution and the desired sensitivity automatically. For example, the processing device 140 may pre-determine a corresponding relationship (e.g., a table) between candidate resolutions, candidate sensitivities, and candidate information relating to candidate subjects, and determine the desired resolution and the desired sensitivity based on the corresponding relationship and the information relating to the target subject, such as, by looking up the table.

In some embodiments, the desired resolution and the desired sensitivity may be set by a user.

In 1304, the processing device 140 (e.g., the generation module 920) may determine reference values of structure parameters of collimating units in a collimator of the imaging apparatus based on the desired resolution and the desired sensitivity.

The reference values of the structure parameters are structure parameter values that can achieve the desired resolution and the desired sensitivity determined without taking the different types of collimating units into consideration. For example, a collimator that includes a single type of collimating units with the reference values of the structure parameters can achieve the desired resolution and the desired sensitivity.

The structure parameters of the collimating units may include an aperture size of a hole in the collimating unit, a hole spacing, a hole channel length, an opening angle, a count of the hole(s) in the collimating unit, a distribution or arrangement of the hole(s) in the collimating unit, a distance between the collimating unit and the center of the accommodating space, or the like, or any combination thereof. More descriptions regarding the structure parameters may be found elsewhere in the present disclosure. See, e.g., FIG. 2 and relevant descriptions thereof.

In some embodiments, a resolution and a sensitivity of the imaging apparatus may be affected by the collimator and a detector of the imaging apparatus.

For example, for a pinhole collimator, the resolution (e.g., a spatial resolution) of the imaging apparatus in a full width at half maximum (FWHM) may be determined according to Equation (10):

$${R}_{s y s} = \sqrt{{( \frac{l}{b} {R}_{det} )}^{2} + {( \frac{l + b}{l} {d}_{eff , R} )}^{2}} , \qquad ( 10 )$$

where R_{sys} refers to the resolution of the imaging apparatus in the FWHM, b refers to a distance from a source (i.e., the target subject) to the collimator, l refers to a distance from the collimator to the detector, R_{det} refers to an intrinsic resolution of the detector, and d_{eff,R} refers to a resolution-effective pinhole diameter, which is defined as Equation (11):

$${d}_{eff , R} = d + \frac{log 2}{μ} tan \frac{a}{2} , \qquad ( 11 )$$

where d refers to a physical pinhole diameter (i.e., an aperture size), μ refers to a linear attenuation coefficient of the material of the collimator (e.g., a main body), and α refers to an opening angle of the pinhole.

The sensitivity of the imaging apparatus may be determined according to Equation (12):

$$g = \frac{{d}_{eff , g}^{2}}{16 {b}^{2}} {cos}^{3} θ , \qquad ( 12 )$$

where g refers to the sensitivity of the imaging apparatus, θ refers to an incident angle of a gamma ray, and d_{eff,g} refers to a sensitivity-effective pinhole diameter, which is defined as Equation (13):

$${d}_{eff , g} = \sqrt{d ( d + \frac{2}{μ} tan \frac{a}{2} ) + \frac{2}{{μ}^{2}} {tan}^{2} \frac{a}{2}} . \qquad ( 13 )$$

According to Equations (10)-(13), the processing device 140 may determine the reference values of the structure parameters of the collimator that can achieve the desired resolution and the desired sensitivity.

In 1306, the processing device 140 (e.g., the generation module 920) may determine target values of the structure parameters and counts of different types of collimating units based on the reference values of the structure parameters.

The target values of the structure parameters are structure parameter values that can achieve the desired resolution and the desired sensitivity determined when taking the different types of collimating units into consideration. For example, a collimator that includes multiple types of collimating units with the target values of the structure parameters can achieve the desired resolution and the desired sensitivity.

In some embodiments, the processing device 140 may determine the target values of the structure parameters and the counts of different types of collimating units by performing data simulation.

For example, the processing device 140 may determine a plurality of sets of candidate values and candidate counts based on the reference values of the structure parameters. For instance, for an aperture size, an average value of the candidate aperture sizes may be equal to the reference aperture size. As another example, the candidate counts may be determined randomly or based on historical data.

For each set of candidate values and candidate counts, the processing device 140 may obtain a simulated resolution and a simulated sensitivity by performing a simulated scan on the target subject based on the set of candidate values and candidate counts, obtain a first difference between the simulated resolution and the desired resolution, and obtain a second difference between the simulated sensitivity and the desired sensitivity. Then, the processing device 140 may determine the target values of the structure parameters and the counts of different types of collimating units based on the first differences and the second differences of the plurality of sets of candidate values and candidate counts. For example, the processing device 140 may designate a set of the candidate values and candidate counts corresponding to a minimum sum of the first difference and the second difference as the target values of the structure parameters and the counts of different types of collimating units.

By determining the target values of the structure parameters and the counts of different types of collimating units based on the desired resolution and the desired sensitivity, the structure parameters and the counts of different types of collimating units can be adjusted precisely, thereby satisfying imaging requirements and improving the imaging quality. Furthermore, by performing the data simulation, the accuracy of the determination of the target values can be improved and the imaging process can be optimized, further satisfying imaging requirements and improving the imaging quality.

It should be noted that the descriptions of the processes 1000-1300 are provided for the purposes of illustration, and are not intended to limit the scope of the present disclosure. For persons having ordinary skills in the art, various variations and modifications may be conducted under the teaching of the present disclosure. For example, the processes 1000-1300 may be accomplished with one or more additional operations not described, and/or without one or more of the operations discussed. Additionally, the order in which the operations of the processes 1000-1300 are not intended to be limiting. However, those variations and modifications may not depart from the protection of the present disclosure.

FIGS. 18A and 18B are schematic diagrams illustrating an exemplary process for super-sampling according to some embodiments of the present disclosure.

Traditional dual-head and triple-head SPECT systems require the rotation of gamma cameras around the patient to acquire scan data (e.g., projections) from different view angles. Each camera head captures gamma rays from a specific direction as it rotates, which is crucial for reconstructing 3D tomographic images.

In contrast, an imaging apparatus with a ring configuration can acquire projections from all angles simultaneously without the need for camera rotation, greatly improving acquisition speed and sensitivity.

In some embodiments, a collimator of the imaging apparatus may be caused to rotate in finer steps. For example, during a scan of a target subject, a collimator of the imaging apparatus may be caused to rotate relative to a detector of the imaging apparatus around a central axis of the imaging apparatus by a preset step size one or more times, and the preset step size may be determined such that each time the collimator rotates, projections of at least a portion of collimating units (e.g., the pinhole 1, a pinhole 2) in the collimator on the detecting modules are shifted by a distance smaller than the bin size of the detector. An acquisition mode corresponding to the finer step size (e.g., smaller than the bin size of the detector) may be also referred to as a super-sampling mode. At this time, the rotation may be also referred to as a sub-pixel rotation, and the super-sampling mode may be also referred to as a sub-pixel mode.

As shown in FIGS. 18A and 18B, a step rotation of a collimator 1810 may cause a relative movement of the collimator 1810 to a detector (e.g., a detector 1, a detector 2). Projections on the detector 1 that radiation rays pass through the pinhole 1 may move from a region AB to a region CD, and the shift of the projections may be a half of a bin size (i.e., an eighth of the region AB). Furthermore, referring to FIG. 8, by rotating the collimating modules 820, an FOV may also rotate slightly, such as, rotate from a shadow region 842 to another shadow region 844.

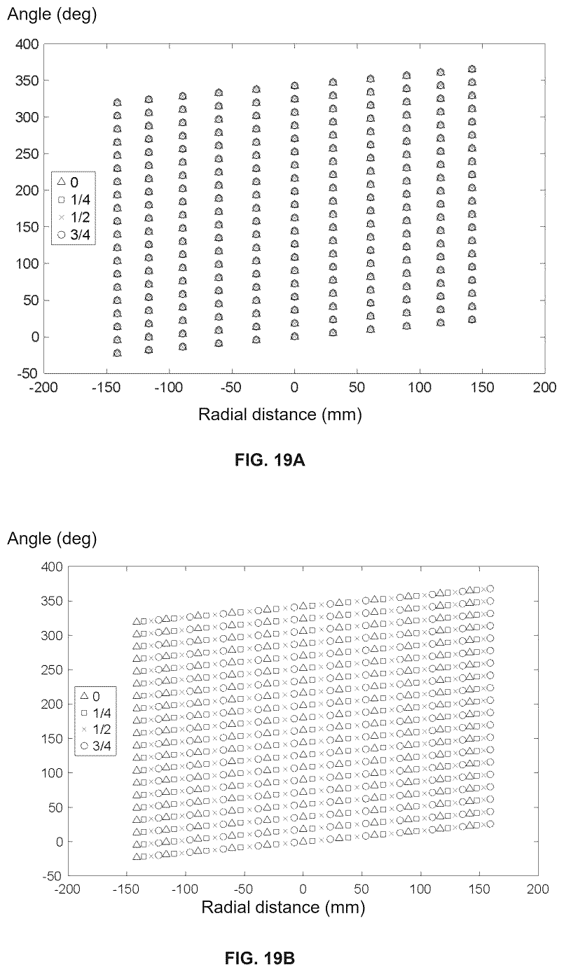

In some embodiments, by performing the super-sampling on the target subject, the collimator is located at multiple relative positions with respect to the detector, scan sets corresponding to the multiple relative positions may be obtained. A data volume of the scan sets collected in the super-sampling mode may be larger than a data volume of scan data collected in a traditional sampling mode (e.g., rotating the collimator and the detector simultaneously). For example, referring to FIGS. 19A and 19B, FIG. 19A is a schematic diagram illustrating an exemplary sampling pattern in sinogram space for a traditional sampling mode according to some embodiments of the present disclosure. FIG. 19B is a schematic diagram illustrating an exemplary sampling pattern in sinogram space for a super-sampling mode according to some embodiments of the present disclosure. As shown in FIGS. 19A and 19B, more detailed projections can be collected in the super-sampling mode than the traditional sampling mode.

By rotating the collimator in the finer steps, the spatial sampling can be effectively increased beyond the intrinsic limitations of detector bin sizes, and a sub-pixel scan can be performed on the subject. This can provide a cost-effective and efficient solution to achieve super-resolution images, comparable to those obtained using more expensive detector technologies like CZT detectors.

In some embodiments, a target reconstruction image may be generated by reconstructing the scan sets in a similar manner as how the target reconstruction image is generated as described in operation 1004.

For illustration purposes, the reconstruction of scan data (also referred to as super-sampled data) collected by an imaging apparatus through super-sampling is taken as an example and described below. The super-sampled data may include super-sampled data sets. Traditionally, an iterative back projection (IBP) algorithm is used to estimate high-resolution images by iteratively refining initial estimated images based on back projected errors between observed and estimated images. To more accurately account for Poisson statistics, a super-sampling reconstruction algorithm based on Maximum Likelihood Expectation Maximization (MLEM) may be introduced. By modeling the probability of observing the super-sampled data as a Poisson distribution, a likelihood function of parameters corresponding to the high-resolution image to be reconstructed may be obtained. Using an Expectation Maximization (EM) strategy, the high-resolution image may be iteratively estimated by first calculating the expected log-likelihood in an expectation step, and then maximizing the log-likelihood in a maximization step to derive an update equation that progressively improves the reconstruction with each iteration.

The expectation of the super-sampled data gₘ may be related to the target subject through Equation (14):

$${\overline{g}}_{m} = {H}_{m} f + {r}_{m} , m = 0 , 1 , … , M - 1 , \qquad ( 14 )$$

where m refers to one super-sampling acquisition among the super-sampling, g̅ₘ refers to a super-sampled data set corresponding to the super-sampling acquisition m, Hₘ refers to tomographic forward projector or system matrix of the super-sampling acquisition m, and rₘ refers to background events (e.g., random and scattered events) of the super-sampling acquisition m. Hₘ and rₘ may be determined by data simulation.

The processing device 140 may rearrange Equation (14) into a matrix form, so as to obtain Equation (15):

$$[ \begin{matrix}{\overline{g}}_{0} \\ {\overline{g}}_{1} \\ ⋮ \\ {\overline{g}}_{M - 1}\end{matrix} ] = [ \begin{matrix}{H}_{0} \\ {H}_{0} \\ ⋮ \\ {H}_{M - 1}\end{matrix} ] f + [ \begin{matrix}{r}_{0} \\ {r}_{1} \\ ⋮ \\ {r}_{m - 1}\end{matrix} ] . \qquad ( 15 )$$

Equation (15) may represent a system model for scan data acquisition and system matrix. Correspondingly, the iterative image reconstruction is performed for the super-sampling according to Equation (16):

$${\hat{f}}^{( k + 1 )} = \frac{{\hat{f}}^{( k )}}{{\sum }_{m = 0}^{M - 1} {H}_{m}^{T} 1} {\sum }_{m = 0}^{M - 1} {H}_{m}^{T} \frac{{g}_{m}}{{H}_{m} {\hat{f}}^{( k )} + {r}_{m}} , \qquad ( 16 )$$

where

$${H}_{m}^{T} 1$$

refers to a sensitivity map for the super-sampling acquisition m. Assuming that the k^{th} iteration is the previous iteration, \hat{f}^{(k+1)} can be regarded as the combined reconstruction image of the current iteration as described above, \hat{f}^{(k)} can be regarded as the combined reconstruction image of the previous iteration as described above.

By combining the finer scan data and using the super-sampling reconstruction algorithms, the imaging resolution can be improved, thereby generating the target reconstruction images with greater detail and precision, which is equivalent to acquiring the scan data at a higher resolution or using a smaller bin size of the detector. Furthermore, the super-sampling can offer improved diagnostic capabilities and better resolution for both whole-body and focused FOV imaging applications.

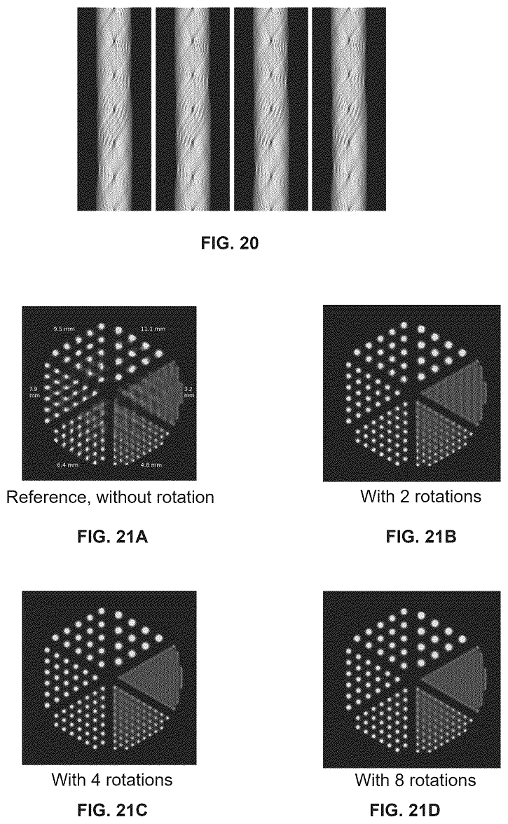

In some embodiments, the rotation times may also affect the sensitivity and resolution of the imaging apparatus. For example, as shown in FIGS. 20 and 21A-21D, images (indicating data in sinogram format) and reconstructions images corresponding to four different rotations of a collimator show that as an increase in the rotations, the image performance (e.g., the resolution) is improved. However, when the rotations exceed a threshold rotation, the improvement in the image performance is diminished.

By introducing the super-sampling mode, the preset step size (or the relative movement of the detector to the collimator) can achieve projection shift smaller than the bin size of the detector, and more detailed projections can be collected, thereby improving resolution in reconstructed images generated based on the scan data (e.g., projections).

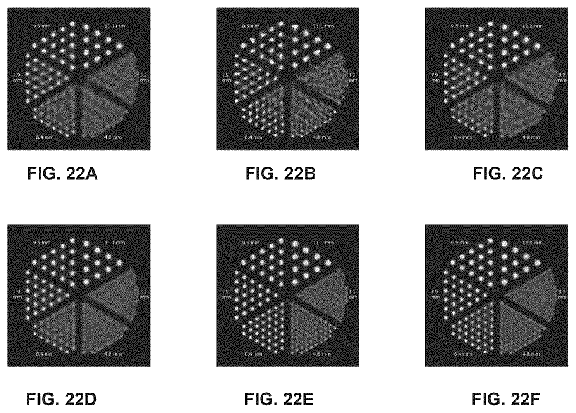

For example, as shown in FIGS. 22A-22C, points in a reconstruction image in FIG. 22A are circular but fuzzy (high sensitivity but low resolution), points in a reconstruction image in FIG. 22B are clear but irregular (high resolution but low sensitivity), and points in a reconstruction image in FIG. 22C are relatively circular and clear (relatively high resolution and sensitivity), wherein the reconstruction image in FIG. 22A is generated based on scan data (with a low count of radiation events) obtained through large pinholes, the reconstruction image in FIG. 22B is generated based on scan data (with a low count of radiation events) obtained through small pinholes, and the reconstruction image in FIG. 22C is generated based on scan data (with a low count of radiation events) obtained through hybrid pinholes. That is, the scan data which is collected through the hybrid pinholes (or the hybrid collimator) can improve the trade-off between the resolution and the sensitivity. As still another example, as shown in FIGS. 22D-22F, points in a reconstruction image in FIG. 22D are circular but fuzzy (high sensitivity but low resolution), points in a reconstruction image in FIG. 22E are clear but irregular (high resolution but low sensitivity), and points in a reconstruction image in FIG. 22F are relatively circular and clear (relatively high resolution and sensitivity), wherein the reconstruction image in FIG. 22D is generated based on scan data (with a high count of radiation events) obtained through large pinholes, the reconstruction image in FIG. 22E is generated based on scan data (with a high count of radiation events) obtained through small pinholes, and the reconstruction image in FIG. 22F is generated based on scan data (with a high count of radiation events) obtained through hybrid pinholes.

Some embodiments of the present disclosure also provide an imaging system. The imaging system may include an imaging apparatus. The imaging apparatus may include a detector including detecting modules arranged along a circumference direction of the imaging apparatus and configured to form an accommodation space, and a collimator including collimating modules arranged within the accommodation space along the circumference direction. The collimating modules may include collimating units. The imaging apparatus may be configured to scan a target subject. During the scan of the target subject, at least a portion of the collimating units may be in an effective state for allowing radiation rays from the target subject to pass through and irradiate the detecting modules, the collimator may be caused to rotate relative to the detector around a central axis of the imaging apparatus by a preset step size one or more times, and the preset step size may be determined such that each time the collimator rotates, projections of the at least a portion of the collimating units on the detecting modules are shifted by a distance smaller than a bin size of the detector. That is, the rotation of the collimator may be a sub-pixel rotation, and the scan of the target subject may be a sub-pixel scan.

In some embodiments, the detector may remain stationary when the collimator is caused to rotate relative to the detector. Alternatively, the collimator may remain stationary when the detector is caused to rotate relative to the collimator.

In some embodiments, the collimating units may have a same type of collimating units or different types of collimating units. For example, different types of collimating units may have different configurations. More descriptions regarding the collimating units and/or the detecting modules may be found elsewhere in the present disclosure. See, e.g., FIGS. 2-22F and relevant descriptions thereof.

It should be noted that the foregoing descriptions are merely provided for the purpose of illustration and are not intended to limit the scope of the present disclosure. For those skilled in the art, amendments and variations may be made under the teaching of the descriptions of the present disclosure. However, these amendments and variations do not depart from the scope of the present disclosure. For example, the super-sampling mode may be performed by rotating the detector. As another example, the super-sampling mode may be performed by rotating the detector and the collimator together, while the detector has a relative rotation or translation with respect to the collimator.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations thereof, are not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may further be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the count of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Therefore, by way of example, but not of limitation, alternative configurations of the embodiments of the application may be utilized in accordance with the teachings herein. Accordingly, embodiments of the present application are not limited to that precisely as shown and described.

What is claimed is:

1. An imaging system, comprising an imaging apparatus, the imaging apparatus comprising:
a detector including detecting modules arranged along a circumference direction of the imaging apparatus and configured to form an accommodation space; and
a collimator including collimating modules arranged within the accommodation space along the circumference direction, the collimating modules including collimating units, wherein
the imaging apparatus is configured to scan a target subject,
during the scan of the target subject, at least a portion of the collimating units are in an effective state for allowing radiation rays from the target subject to pass through and irradiate the detecting modules, and
the at least a portion of the collimating units include multiple types of collimating units.

2. The imaging system of claim 1, wherein the multiple types of collimating units are arranged on the same collimating module.

3. The imaging system of claim 1, wherein the multiple types of collimating units are arranged on different collimating modules.

4. The imaging system of claim 1, wherein the multiple types of collimating units have different aperture sizes.

5. The imaging system of claim 4, wherein
the multiple types of collimating units include a first collimating unit and a second collimating unit, the first collimating unit has a first aperture size, the second collimating unit has a second aperture size smaller than the first aperture size,
a first distance between the first collimating unit and a central axis of the imaging apparatus is the same as a second distance between the second collimating unit and the central axis of the imaging apparatus, and the scan is a single-FOV scan.

6. The imaging system of claim 4, wherein
the multiple types of collimating units include a first collimating unit and a second collimating unit, the first collimating unit has a first aperture size, the second collimating unit has a second aperture size smaller than the first aperture size,
a first distance between the first collimating unit and a central axis of the imaging apparatus is different from a second distance between the second collimating unit and the central axis of the imaging apparatus, and the scan is a dual-FOV scan.

7. The imaging system of claim 6, wherein
the dual-FOV scan has a first FOV formed by the first collimating unit and a second FOV formed by the second collimating unit,
the target subject includes a region of interest (ROI),
the first FOV covers the target subject, and the second FOV covers the ROI.

8. The imaging system of claim 6, wherein the dual-FOV scan has a first FOV formed by the first collimating unit and a second FOV formed by the second collimating unit,
the imaging system further comprises a processing device configured to:
obtain scan data collected by the detector during the scan;
generate a target reconstruction image of the target subject by reconstructing the scan data, wherein the target reconstruction image includes a second portion corresponding to the second FOV and a first portion other than the second portion, the voxel size of voxels in the second portion is smaller than the voxel size of voxels in the first portion.

9. The imaging system of claim 4, wherein the multiple types of collimating units include a first collimating unit and a second collimating unit, the first collimating unit has a first aperture size, the second collimating unit has a second aperture size smaller than the first aperture size, and the imaging system further comprises a processing device configured to:
obtain scan data collected by the detector during the scan, the scan data includes first scan data corresponding to the first collimating unit and second scan data corresponding to the second collimating unit;
generate an initial reconstruction image of the target subject based on the first scan data; and
generate a target reconstruction image of the target subject by updating the initial reconstruction image based on the second scan data.

10. The imaging system of claim 1, further comprising a processing device configured to:
obtain scan data collected by the detector during the scan, the scan data includes multiple data subsets corresponding to the multiple types of collimating units;
generate a target reconstruction image of the target subject by reconstructing the scan data through an iterative process including iterations, a current iteration including:
for each type of collimating unit, generating an intermediate reconstruction image based on the data subset corresponding to the type of collimating unit and a combined reconstruction image of a previous iteration;
generating a combined reconstruction image of the current iteration by combining the intermediate reconstruction images of the multiple types of collimating units based on weights of the multiple types of collimating units;
proceeding to a next iteration or designating the combined reconstruction image of the current iteration as the target reconstruction image.

11. The imaging system of claim 10, wherein the weight of a type of collimating unit is determined based on a sensitivity map of the type of collimating unit.

12. The imaging system of claim 10, wherein the weight of a type of collimating unit is determined based on a scaling factor corresponding to the type of collimating unit, the scaling factor is associated with the count of radiation events detected by the detector in the scan.

13. The imaging system of claim 10, wherein the weights of the multiple types of collimating units are determined based on scaling factors of the multiple types of collimating units, and the scaling factors are determined by:
obtaining candidate reconstruction images corresponding to candidate sets each of which includes candidate scaling factors of the multiple types of collimating units;
determining a contrast recovery coefficient (CRC) and a signal-to-noise ratio (SNR) of each of the candidate reconstruction images;
determining the scaling factors based on the CRC and the SNR of each of the candidate reconstruction images.

14. The imaging system of claim 1, wherein during the scan of the target subject, the collimator is caused to rotate relative to the detector around a central axis of the imaging apparatus by a preset step size one or more times.

15. The imaging system of claim 14, wherein the preset step size is determined such that each time the collimator rotates, projections of the at least a portion of the collimating units on the detecting modules are shifted by a distance smaller than a bin size of the detector.

16. The imaging system of claim 1, further comprising a processing device configured to:
determine a desired image resolution and a desired sensitivity of the imaging apparatus based on information relating to the target subject;
determine reference values of structure parameters of the collimating units based on the desired image resolution and the desired sensitivity; and
determine, based on the reference values of the structure parameters of the collimating units, target values of the structure parameters and counts of the different types of collimating units.

17. A method for medical imaging implemented on a computing device having at least one processor and at least one storage device, the method comprising:
obtaining scan data collected by a detector of an imaging apparatus during a scan of a target subject; and
generating a target reconstruction image of the target subject by reconstructing the scan data, wherein
the scan data are collected by the detector by detecting radiation rays from the target subject that pass through at least a portion of collimating units of a collimator of the imaging apparatus,
the at least a portion of the collimating units includes multiple types of collimating units in different configurations.

18. The method of claim 17, wherein the scan data includes multiple data subsets corresponding to the multiple types of collimating units, and the generating a target reconstruction image of the target subject comprises:
for each type of collimating unit, generating an intermediate reconstruction image based on the data subset corresponding to the type of collimating unit and a combined reconstruction image of a previous iteration;
generating a combined reconstruction image of the current iteration by combining the intermediate reconstruction images of the multiple types of collimating units based on weights of the multiple types of collimating units;
proceeding to a next iteration or designating the combined reconstruction image of the current iteration as the target reconstruction image.

19. An imaging system, comprising an imaging apparatus, the imaging apparatus comprising:
a detector including detecting modules arranged along a circumference direction of the imaging apparatus and configured to form an accommodation space; and
a collimator including collimating modules arranged within the accommodation space along the circumference direction, the collimating modules including collimating units, wherein
the imaging apparatus is configured to scan a target subject,
during the scan of the target subject,
at least a portion of the collimating units are in an effective state for allowing radiation rays from the target subject to pass through and irradiate the detecting modules,
the collimator is caused to rotate relative to the detector around a central axis of the imaging apparatus by a preset step size one or more times, and
the preset step size is determined such that each time the collimator rotates, projections of the at least a portion of the collimating units on the detecting modules are shifted by a distance smaller than a bin size of the detector.

20. The imaging system of claim 19, wherein the detector remains stationary when the collimator is caused to rotate relative to the detector.
